SEQUENCE LISTING (2 sequences)

SEQ ID NO: 1 - protein, 55 aa, Artificial Sequence
  SEQ ID NO 1 represents the amino acid sequence of carboxylated osteocalcin (Gla=gamma-Carboxyglutamic Acid; Disulfide bridge 23-29) Molecular weight = 5929.5
YLYQWLGAPVPYPDPLGLAPRRGLAVCGLALNPDCDELADHIGFQEAYRRFYGPV

SEQ ID NO: 2 - protein, 42 aa, Artificial Sequence
  SEQ ID NO 2 represents amino acid sequence for Abeta42 with Tottori mutation
DAEFRHNSGYEVHHQKLVFFAEDVGSNKGAIIGLMVGGVVIA

(19) United States
(12) Patent Application Publication
(10) Pub. No.: US 2022/0175888 A1
(43) Pub. Date: Jun. 9, 2022

(54) CARBOXYLATED OSTEOCALCIN FOR TREATMENT OF AMYLOIDOSIS OR DISEASES ASSOCIATED WITH ABNORMAL PROTEIN FOLDING

(71) Applicant: NATIONAL INSTITUTE OF IMMUNOLOGY, New Delhi (IN)

(72) Inventors: Sarika GUPTA, New Delhi (IN); Viji VIJAYAN, New Delhi (IN); Ibrar Ahmed SIDDIQUE, New Delhi (IN)

(21) Appl. No.: 17/051,272

(22) PCT Filed: Apr. 30, 2019
(86) PCT No.: PCT/IN2019/050345

§ 371 Date: Oct. 28, 2020

(30) Foreign Application Priority Data
Apr. 30, 2018 (IN) 201811016306

(51) Int. Cl. A61K 38/22; A61K 47/54; A61P 25/28

(52) U.S. Cl. CPC A61K 38/22; A61P 25/28; A61K 47/542

(57) ABSTRACT

The present disclosure relates to improved compositions that are effective in management of disorders caused by pathogenic amyloid deposits. The disclosure discloses a composition comprising carboxylated osteocalcin which is effective in therapeutic clearance of abnormal amyloid deposits.

### FIELD OF INVENTION

The present disclosure broadly relates to the field of treatment of diseases. In particular, it relates to use of carboxylated osteocalcin or Gla-OC to treat amyloidosis, method to clear abnormal amyloid deposits, tangles or abnormal proteinaceous materials, and use of Gla-OC to attenuate blood brain barrier disruption

### BACKGROUND OF INVENTION

The Amyloid diseases: Amyloids are aggregates of globular proteins that get folded into a certain shape, which permits many copies of that particular protein to associate or aggregate together and form cross-β structures called amyloid fibrils. This happens when a protein loses its normal conformation and physiological functions. The formation of amyloid fibres typically accompanies a disease and each disease is characterized by a specific peptide or protein that aggregates. Thus, amyloid fibril formation and deposition can cause various clinical complications in the human body and these disorders are collectively referred to as amyloid diseases or “amyloidoses”. Today amyloidoses represent a large group of diseases, conformational changes and pathogenic aggregation propensity have been identified for around 24 globular proteins. All these amyloidogenic proteins typically possess: (a.) reduced folding stability under specific conditions; (b.) strong propensity to acquire more than one conformation; and (c.) the capacity to form fibrillar structures. It has also been identified that many of the disease-associated amyloidogenic proteins have extensive regions of intrinsic disorder in their free soluble forms and have specific, short internal amino acid sequences required to support aggregation. The formation of an aggregation-prone state is triggered by many reasons. These include mutations, proteolytic cleavage, or a seeding. Examples are as follows: (a.) mutations in genes encoding amyloid precursor protein, islet amyloid polypeptide, alpha-synuclein, Huntington, prion protein and transthyretin proteins cause Alzheimer's disease (AD), type 2 diabetes, Parkinson's disease (PD), Huntington's disease, Creutzfeldt-Jakob disease and familial adenomatous polyposis respectively. (b.) seeding of fibrillar proteins of serum amyloid A (SAA) derived from the amyloid A (AA) amyloidosis animal model to susceptible recipient induces AA amyloidosis. (c.) Proteolytic cleavage of Ser52Pro variant transthyretin triggers amyloid aggregation and fibrillization [Westermark P, Westermark GT. 2013. Seeding and cross seeding in amyloid diseases. Proteopathic seeds and cross-seeding in amyloid diseases. In: Jucker M., Christen Y. (eds) Proteopathic Seeds and Neurodegenerative Diseases. Research and Perspectives in Alzheimer's Disease. Springer, Berlin, Heidelberg. Pp. 44-60; Mangione P P, Porcari R, Gillmore J D, Pucci P, Monti M, Porcari M, Giogetti S. et al. 2014. Proc Natl Acad Sci USA. 111: 1539-1544].

Once formed, the aggregated proteins are thermodynamically stable because of the extensive contacts made between the protein chains of the polymer. The thermodynamic stability then confers the aggregates an ability to “propagate” as well. Propagation is a key feature of misfolded proteins that allows protein aggregates to spread in a prion like manner by recruiting normally folded counterparts to adopt pathogenic conformations. Pathogenic amyloids then spread from cell to cell to initiate new pathology via activity dependent secretion by exosomes and/or chaperone-mediated pathways. Misfolding of one disease causing protein can induce misfolding of other aggregation prone proteins and hence aggregates of different disease proteins may be found in the same patient suffering from amyloid disease. The accumulation of the protein itself can hamper the proteostatic network and trigger the misfolding of unrelated proteins that fold normally otherwise. Under normal conditions, any abnormal protein aggregates formed due to misfolding are degraded by autophagy or by proteasomal machinery. The pathway by which a protein is degraded (ubiquitin proteosome machinery versus autophagy) varies depending on whether the protein is soluble or fibrillar in state and the post translational modifications it bears. But in amyloid diseases, the protein aggregates are highly resistant to degradation since proteosomes can degrade only single chain polypeptide chains in partially or fully unfolded conformation.

Protein aggregates formed during amyloidosis are toxic. In particular, pre-fibrillar aggregates are most noxious to cells. Though how the aggregates cause cellular toxicity is still elusive, it is presumed that aggregates act primarily by toxic gain of function and/or dominant negative effects, though loss of function have also been observed. Examples for these effects include interference of synaptic signalling by misfolded amyloid beta, tau and alpha-synuclein, disruption of microtubule function and cellular transport by mutant tau and inhibition of mitochondrial protein import by alpha-synuclein. Other examples include the following: (a.) in type 2 diabetes, pancreatic islet amyloid deposits consisting of aggregated islet amyloid polypeptide or IAPP (amylin) causes beta-cell toxicity and failure; (b.) in amyloid light chain (AL) amyloidosis or primary amyloidosis (that occur with bone marrow cancer), a plasma cell disorder, aggregation of immunoglobulin components (L-chain) causes toxicity to kidneys, heart, gastrointestinal tract, spleen, endocrine glands, skin, lungs and liver; (c.) in secondary amyloidosis conditions like rheumatoid arthritis, familial Mediterranean fever, osteomyelitis or granuloma iletis, increased production of acute phase protein forms amyloid cause kidney toxicity. In familial amyloidosis, transthyretin or TTR amyloids causing neuropathy or cardiomyopathy; and (d.) in beta-2-microglobulin amyloidosis (found in patients with chronic renal failure) amyloid deposit of beta-2-microglobulin around joints cause cellular toxicity.

Amyloidosis in neurodegenerative diseases: Neurodegenerative diseases are basically disorders that affect brain and central nervous system that involve neuronal loss. Although the name “neurodegenerative” suggests deterioration of neurons, accruing evidence suggests that these are not merely diseases of dying neurons. Non-neuronal cells in the brain, such as glial cells, which are even more abundant in the brain and the central nervous system than neurons also play major roles in disease progression. Some of the important examples of neurodegenerative diseases are Alzheimer's disease, Parkinson's disease, Huntington's disease, amyotrophic lateral sclerosis, frontotemporal dementia and spinocerebellar ataxias. Some of the publications that describe the common features of neurodegenerative disorders are listed here: [Dale E. Bredesen, Rammohan V. Rao and Patrick Mehlen. Cell death in the nervous system. Nature 443 (2006): 796-802; Christian Haass. Initiation and propagation of neurodegeneration. Nature Medicine 16 (2010): 1201-1204; Michael T. Lin and M. Flint Beal. Mitochondrial dysfunction and oxidative stress in neurodegenerative diseases. Nature 443 (2006) 787-795].

There are many causes for neurodegenerative diseases. These include genetic, protein misfolding, alterations in protein degradation machinery, changes in axonal transport, mitochondrial dysfunction and programmed cell death. Among the above-mentioned reasons, protein misfolding is an important phenomenon, which is widely investigated. Hence, neurodegenerative disorders are also referred to as ‘neurodegenerative proteinopathy’. Table 1 comprise a list of aggregation prone proteins and the neurodegenerative diseases it causes. This is depicted from a published report [“Toxic proteins in neurodegenerative disease” by J. Paul Taylor et al. Science Magazine Vol 296. pp. 1991-1995 (Jun. 14, 2002)]

TABLE 1

| | Protein | Toxic | | Risk |
|---|---|---|---|---|
| Disease | deposits | protein | Genes | factor |
| Alzheimer's | Extracellular | Abeta | APP | ApoE4 |
| Disease | Plaques | Tau | Presenilin1 | allele |
| | Intracellular | | Presenilin2 | |
| | tangles | | | |
| Parkinson's | Lewis bodies | Tau | Alpha- | Tau |
| disease | | Alpha- | synuclein | linkage |
| | | synuclein | Parkin | |
| | | | UCHL | |
| Prion | Prion plaque | PrP^{5c} | PRNP | Homozygosity |
| | | | | at prion |
| | | | | codon 129 |
| Poly- | Nuclear and | Poly- | 9 different | |
| glutamine | cytoplasmic | glutamine | genes with | |
| | inclusions | containing | CAG repeat | |
| | | proteins | expansion | |
| Taupathy | Cytoplasmic | Tau | Tau | Tau |
| | tangles | | | linkage |
| Familial | Bunia bodies | SOD1 | SOD1 | |
| Amyotrophic | | | | |
| lateral | | | | |
| sclerosus | | | | |

Although some aspect of each of the neurodegenerative disorder mentioned in the table is different, the pathology and symptoms that these have are common which often makes therapeutic strategies similar. A reference that shows the overlap of proteinopathy is described here:

The diagram adapted from Molecular Degeneration by Moussaud et al. (2014) explains the following: In numerous neurodegenerative disorders, amyloid deposits composed of alpha-synuclein protein (red circle), tau protein (blue circle) and Abeta peptide (yellow circle) have been identified. The pathologies are not hermetically isolated categories but form a range and concomitance of alpha-synuclein and tau pathology is not rare. For example, alpha-synuclein pathology (or synucleinopathy) is not restricted to PD but is a feature of numerous dementing disorders such as pervasive developmental disorder, dementia with Lewis bodies and frequently occurs in AD where it contributes to secondary symptoms. By contrast tauopathy is repeatedly observed in numerous disorders primarily classified as synucleinopathies and may contribute to clinical heterogeneity [Moussaud S, Jones D R, Moussaud-Lamodiere E, Delenclos M, Ross O A, McLean P J. 2014. Alpha-synuclein and tau: teammates in neurodegeration? Molecular Neurodegeration. 9: 43]. There are also examples to cite that Abeta deposition in brain occurs in other neurodegenerative diseases other than A D. Mastaglia et al. (2003) reported vascular deposition of Abeta in the brain cortex of P D patients [Mastglia F L, Johnsen R D, Byrnes M L, Kakulas B A. 2003. Prevalence of amyloid-beta deposition in the cerebral cortex in Parkinson's disease. Mov Disord. 18: 81-86.]. The relationship between corticostriatal Abeta-amyloid deposition and cognitive dysfunction in a cohort of patients with P D at risk for dementia was investigated Petrou et al. in 2012 [Petrou M, Bohnen N I, Muller M, Koeppe R A, Albin R, Frey K. 2012. Abeta-amyloid deposition in patients with Parkinson disease at risk factor for development of dementia. Neurology. 79: 1161-1167]. In view of the above mentioned facts it may be contemplated that although the patent focusses on AD, the invention described is fully applicable to any disease exhibiting deposition of amyloid fibrils and toxicity.

Alzheimer's Disease (AD)—Role of amyloid deposits and fibrillary tangles: Among the many neurodegenerative diseases in humans, the common form is AD. AD is a progressive neurodegenerative disorder and growing public health problem among the elderly. According to World Health Organization (WHO), AD is the most common cause of dementia, accounting for as many as 60˜70% of senile dementia cases affecting 47.5 million people worldwide in 2015. The median survival time after the onset of dementia ranges from 3.3 to 11.7 years. Age is a risk factor for AD, which is the most common cause of dementia affecting persons aged over 65 years. Over 95% of all AD cases are diagnosed suffering late-onset AD and are aged 65 years and over; only 1˜5% of all cases are early-onset AD. Globally, the incidence rate for AD doubles every five years after the age of 65. As the average age of the population increases, the number of cases of AD is expected to triple by 2050, reaching over 115 million. Available data also shows that by the year 2020, approximately 70% of the world's population aged 60 and above will be suffering from AD, with 14.2% in India [Mathuranath P S, George A, Ranjith N, Justus S, Kumar M S, Menon R, Sarma P S, Verghese J. 2012. Incidence of Alzheimer's disease in India: a 10 years follow-up study. Neurol India. 60: 625-630].

Some of the clinical features of AD include progressive loss of memory and onset of confusion and dementia. Other characters of AD include irritability, aggression, spatial orientation, mood swings and trouble with language. These symptoms progress over a period of 8 to 10 years. The development of AD in patients can be divided into four stages with advancing stages of cognitive and functional impairments. (a.) pre-dementia; (b.) mild early start of the disease; (c.) moderate progressive brain deterioration; (d.) severe or advanced stage where the AD patient is bedridden and completely dependent.

Apart from neuronal loss, accumulation of ‘amyloid plaques’ and ‘neurofibrillary tangles’ in the cortices and hippocampal regions of the brain histologically illustrate AD. Amyloids also accumulate in the lumens and lumen-walls of brain vessels. Similar histologies are also found, for example, in Guam-Parkinsonism dementia complex, Dementia Pugilistica, Parkinson's Disease, adult Down Syndrome, subacute Sclerosing Panencephalitis, Pick's Disease, Corticobasal Degeneration, Progressive Supranuclear Palsy, Amyotrophic Lateral Sclerosis/Parkinsonism Dementia Complex, Hallervorden-Spatz Disease, Neurovisceral Lipid Storage Disease, Mediterranean Fever, Muckle-Wells Syndrome, Idiopathetic Myeloma, Amyloid Polyneuropathy, Amyloid Cardiomyopathy, Systemic Senile Amyloidosis, Hereditary Cerebral Hemorrhage with Amyloidosis, Alzheimer's disease, Scrapie, Creutzfeldt-Jacob Disease, Fatal Familial Insomnia, Kuru, Gerstamnn-Straussler-Scheinker Syndrome, Medullary Carcinoma of the thyroid, Isolated Atrial Amyloid, Beta2-Microglobulin Amyloid in dialysis patients, Inclusion Body Myositis, Beta2-Amyloid deposits in muscle wasting disease, and Islets of Langerhans Diabetes Type2 Insulinoma. The Polyglutamine diseases including Huntington's Disease, Kennedy's Disease, and at least six forms of Spinocerebellar Ataxia involving extended polyglutamine tracts are further examples of such deposits.

The amyloid plaques in AD brain comprise beta-amyloid peptides (Abeta). Under normal conditions, Abeta is a 39-43 amino acid peptide derived from the processing of a larger membrane protein called the beta-amyloid precursor protein (APP). Abeta is a membrane protein that is required for neural growth and repair. Its derivation involves a processing procedure which is a two-step proteolytic process involving beta- and gamma-secretases (presenilin or PS is the sub-component of gamma secretase that is responsible for the cutting of APP). The beta-site APP cleaving enzyme (BACE1), first cleaves APP to generate a membrane bound soluble C-terminal fragment. A succeeding cleavage of the C-terminal fragment by the gamma-secretase activity further generates Abeta40 and Abeta42. Both types of peptide are found in amyloid plaques. Under normal conditions, about 90% of secreted Abeta peptides is Abeta40, which is a soluble form of the peptide which slowly converts to an insoluble beta-sheet configuration and thus is eliminated from the brain. On the other hand, Abeta42 species comprise only 10% of secreted Abeta peptides but has the capacity to exist in different aggregation states. Intracellular assembly states of Abeta42 include monomeric, oligomeric, protofibrillar, and fibrillar states. The monomeric species are not pathological, however the nucleation dependent protein misfolding makes the Abeta42 toxic. To explain how Abeta42 causes AD, researchers have put forth many hypotheses. According to the ‘amyloid hypothesis’, initially proposed by Hardy and Higgins in 1992 and updated by Hardy and Selkoe in 2002 missense mutations in APP or PS1 or PS2 genes causes increased production of Abeta42. The oligomerization of Abeta and its deposition as diffuse plaques directly affects neuronal synapses and/or activates microglia and astrocytes which then causes synaptic and dendritic injury. These also promote release of mediators like complement, cytokines etc. by glial cells which alters kinase phosphatase activities in neuron that subsequently forms toxic intracellular tangles within the neuron. These changes which with time induces neuronal deficit, neuronal death and later dementia. A predominant modern theory states that soluble oligomers of Abeta42, but not monomers or insoluble amyloid fibrils, may be accountable for synaptic dysfunction in the brains of AD patients and in AD animal models. It is now demonstrated that metastable intermediates of Abeta in the formation of fibrils by synthetic Abeta42 referred to as AD diffusable ligands (ADDLs) or protofibrils are injurious to neurons. There are also reports that large polymeric aggregates (such as the amyloid plaques) represent sedentary reservoirs of species, which are in equilibrium with the smaller, putatively neurotoxic assemblies. These different forms of Abeta42 primarily affect neurons in the olfactory bulb and associated brain regions like the entorihinal cortex, hippocampus, amygdaloid nuclei, nucleus basalis of Meynert, locus ceruleus and brain stem raphe nuclei.

The ‘Tau hypothesis’ states that excessive or abnormal phosphorylation of Tau results in the transformation of normal adult Tau into PHF-tau (paired helical filament) and neurofibrillary tangles or NFTs. NFT are aggregates of hyperphosphorylated Tau protein, which normally bind microtubules and assist with their formation and stabilization. When Tau is hyperphosphorylated Tau is unable to bind microtubules causing microtubules to become unstable. Thus, the capacity of Tau to maintain its normal biological function is dependent upon its phosphorylation state. The unbound Tau then aggregates and forms NFTs. Braak staging defines role of NFT in development of AD. In stages I and II, NFT is confined to transentorhinal region of brain whilst in stages III and IV, NFT appears in hippocampus and affects limbic movement [Braak H. et al. Evolution of Alzheimer's disease related cortical lesions. J Neural Transm Suppl. 1998. 54: 97-106]. During stages V and VI NFT appears in large areas of neocortex. Distinct morphological stages of NFT formation have been also distinguished in the AD brain. Each individual stage is associated with specific phosphorylation events that contribute to the evolution of Tau pathology. In a normal neuron, Tau phospho-epitopes pSer262, pThr153 and pThr231 are seen. Mature NFTs immunostain Tau epitopes of pThr175/181, pSer46, pSer214, pSer262/pSer356 and pSer422 and demonstrate dense filamentous aggregates of cytoplasmic Tau that perpetually displaces the cell nucleus towards the periphery of the soma. Upon death of the neuron, extracellular ‘ghost NFTs’ appear that comprise considerable amounts of filamentous Tau protein. Extracellular tangles immunostain AT8 and PHF1 recognizing pSer202/pThr205 and pSer396/pSer404, respectively. It is also proposed that phosphorylation at Thr231 is an initiating event for the formation of NFTs, trailed by oligomeric tau aggregation, filament formation and neuronal cell death.

Other than AD, Tau phosphorylation is also seen alongside other neurodegenerative disorders like Parkinson's Disease or PD. In PD, mutations in the MAPT (microtubule associated protein Tau) gene viz. MAPT splice-site and missense mutations such as G272V, N279K, P301L, V337M and R406W causes frontotemporal dementia with parkinsonism-17 FTDP-17 T. Mutations such as P301L and N279K primarily cause familial frontotemporal dementia or FTD whilst 5305N mutation incites FTD with minimal Parkinsonism. The K3691 mutation is responsible for L-DOPA sensitive Parkinsonism whilst deltaN296 mutation causes familial atypical progressive supranuclear palsy. Research has shown that tau alone is sufficient to provoke severe neurodegeneration leading to Parkinsonism.

‘Vascular hypothesis’ for AD states that presence of vascular risk factors create a ‘Critically Attained Threshold of Cerebral Hypoperfusion’ (CATCH) affecting protein synthesis, development of plaques, inflammatory response and synaptic damage leading to the manifestations of AD.

In the ‘inflammatory hypothesis’, glial cells like microglia and astrocytes and to a lesser extent, neurons incite an inflammatory cascade in AD. Microglial cells get activated by Abeta42 via cell surface expression of major histocompatibility complex II (MHC II) that elicits secretion of the pro-inflammatory cytokines and chemokines like interleukin-1β, interleukin-6, tumor necrosis factor α, interleukin-8, macrophage inflammatory protein-la and monocyte chemoattractant protein-1. Abeta elicits phagocytic response in microglia and expression of nitric oxide synthase (NOS) resulting in neuronal damage. The hypothesis also states that astrocytes around Abeta deposits secrete interleukins, prostaglandins, leukotrienes, thromboxanes, coagulation factors and protease inhibitors that augment AD pathology.

In the ROS hypothesis, reactive oxygen species (ROS) like hydrogen peroxide radicals (H₂O₂), hydroxyl radicals (OH.) and the superoxide radical (O₂⁻.) produced in excess owing to erroneous electron transport chain or ETC damage lipids, proteins, nucleic acids and sugars essential for the structural and functional integrity of neurons. These ROS produced also induces the formation of AGEs or advanced glycation end products which causes lipid peroxidation and amplification of ROS production during AD. AD is also characterized by antioxidant deficit. Reduction in enzyme activities of Cu/Zn SOD (superoxide dismutase) and deficiency of glutathione (GSH) are observed during AD. An antioxidant upregulated during AD is hemoxygenase-1 or HO-1, an inducible enzyme. HO-1 catabolises heme to biliverdin, Fe²⁺ and carbon monoxide (CO). CO protects neurons from oxidative stress induced apoptosis by inhibiting kv2.1 channels that mediate cellular K⁺ efflux as an early step in the apoptotic cascade. There are also reports that state that HO-1 expression is elicited to protect against Abeta toxicity via synthesis of CO and protection occurs via inhibition of AMPK or AMP activated protein kinase pathway.

The other strong hypothesis that demonstrates how AD occurs is the ‘cholesterol hypothesis’, which relates ApoE lipoprotein to AD. Normally, apolipoprotein E4 (ApoE4) play role in metabolism of cholesterol. The epsilon allele of ApoE is major risk factor for AD. During AD ApoE acts as an Abeta binding protein and affects the deposition and clearance of Aβ, and causes amyloid deposition. The involvement of cholesterol is demonstrated by the fact that intracellular cholesterol regulates APP processing by directly modulating secretase activity or by affecting the intracellular trafficking of secretases and/or APP and higher levels of cholesterol increases gamma-secretase activity.

According to the ‘metallobiology hypothesis’, both Abeta and APP have metal ion binding sites and bind and precipitate metals like Cu, Fe and Zn²⁺. Metals like Cu²⁺ and Fe²⁺ gets oxidized upon binding with Abeta and generate H₂O₂ creating a milieu for the generation of highly reactive hydroxyl radicals that can oxidize Abeta-Cu²⁺ and form cross-linked, soluble and degradation resistant forms of Abeta.

In the ‘insulin signaling hypothesis’, abnormal function of the insulin/insulin-like growth factor I (IGF-I) axis like reduced level and decreased sensitivity to these peptides are reasons for AD development. Both stimulate Abeta release from neurons. The release of Abeta into extracellular space by insulin contributes to extraneuronal accumulation of beta-amyloid that competes for insulin degrading enzyme (IDE). On the other hand, IGF-I decreases brain levels of beta-amyloid and increases plasma levels of beta-amyloid complexed to transport proteins ie. IGF-I stimulates clearance of brain beta amyloid. Studies have shown that reduced sensitivity to blood-borne IGF-I at blood brain barrier (BBB) reduces clearance of beta-amyloid, causing brain accumulation of beta-amyloid. A resistant state to insulin/IGF-I in neurons is brought about when high levels of Abeta antagonizes insulin and IGF-I binding to their corresponding receptors facilitating a homeostatic compensatory mechanism whereby levels of insulin/IGF-I increase to rescue loss of function on target cells. In this scenario, insulin also diminishes availability of IDE to degrade β amyloid and as a result more β amyloid accumulates and establishes a self-perpetuating vicious circle.

In the ‘cell cycle hypothesis’ cell cycle control is deranged in AD and ‘vulnerable neurons’ re-enter the cell cycle. APP has role in activation of neuronal cell cycle proteins and a failure of regulation of this pathway occurs during AD. The processes with increased APP include: (a.) an increase in expression of APP-BP1 (APP binding protein I) in lipid rafts which post interaction with APP activates a pathway leading to the conjugation of neddylated proteins like NEDD8 or cullins that promote familial AD or FAD APP-mediated cell cycle entry (through the S-M checkpoint) and apoptosis; (b.) entry of the neurons into the S phase of the cell cycle; and (c.) neuronal apoptosis. Another cell cycle mediator important in AD is GSK3 or glycogen synthase kinase, over-activity of which causes memory impairment, Tau hyper-phosphorylation, increased beta-amyloid production, and inflammatory responses. Activation of PPARγ (peroxisome proliferator activated receptor γ) is also associated in cell cycle as it inhibits the generation of proinflammatory and neurotoxic products in microglia and monocytes exposed to beta-amyloid. Cyclin-dependent kinases (cdk) like cdc2, cdk4, and cdk5 are also associated with Tau hyperphosphorylation and the consequent development of neurofibrillary tangles in AD.

The above-mentioned hypotheses demonstrate the pathogenic complexity of amyloidosis. These also suggest that a combination of therapeutic interventions which impact different stages of amyloidogenic cascade is required for treating deposit disorders like neurodegerativedisease [Bellotti V, Nuvolone M, Giorgetti S, Obici L, Palladini G, Russo P, Lavatelli F, Perfetti V, Merlini G. 2007. The workings of the amyloid diseases. Ann Med. 39: 200-207].

All neurodegenerative diseases involving abnormal amyloid deposition are clinically uncontrollable. Current approved treatments against diseases like AD utilize two strategies; (a.) symptomatic treatment; and (b.) disease modifying treatment. Anti-cholinestrase inhibitors are used as symptomatic treatment, while antioxidants and anti-inflammatory agents are used for disease modifying treatment. All the current treatments are palliative and helps the patient temporarily in slowing disturbances in AD patients. The ongoing clinical trials are still searching for effective drug(s) against AD. Till date protein cleavage inhibitors, post translational modification inhibitors, extrinsic molecular chaperones and activation of endogenous clearance pathways have been used in attempts to manage protein misfolding.

Though tremendous efforts have been carried out in recent years to develop small molecules for inhibiting Abeta aggregation, results of clinical studies indicate that these were merely futile. The review article by Hung and Fu (2017) provides a list of acetylcholinesterase inhibitors, agonists and antagonists of neurotransmitter receptors and beta-secretase (BACE) or gamma-secretase inhibitors targeting Abeta clearance or tau protein, as well as anti-inflammation compounds that flopped at various stages of the clinical trials owing to the numerous adverse effects like liver toxicity and cerebral microbleeds these produced [Hung S, Fu W. 2017. Drug candidates in clinical trials for Alzheimer's disease. Journal of Biomedical Science. 24: 47]. In most cases, small molecule inhibitors fail because of the lack of proper association with Abeta-Abeta interaction surface. While protein-small molecule interaction regions are only 300-1000 Å, protein-protein interactions are approximately 1500-3000 Å that proper steric hindrance is not generated to block Abeta aggregation. Other reasons are as follows: Often the regions of protein-protein interactions are featureless ie. without any grooves or pockets into which a small molecule can dock in an energetically favourable manner. The highly plastic nature of protein surfaces is another factor that upsets inhibition [Nie Q, Du X, Geng M. 2011. Small molecule inhibitors of amyloid beta peptide aggregation as potential therapeutic strategy for Alzheimer's disease. Acta Pharmacologica Sinica. 32: 545-551].

Apart from small molecule inhibitors, some Abeta binding peptide molecules were also designed and tested against Abeta42 aggregation. Examples include beta-sheet breaker peptides, LPYFDA, PPI-1019, Aβ12-28P etc. which showed high specificity, low toxicity and high biological activity but failed in clinical trials due to immunogenicity, poor bioavailability and low blood brain barrier permeability. Monoclonal antibodies (mAb) like Bapineuzumab, Solanezumab, Gantenerumab, Crenezumab, Ponezumab etc. have also been tried and tested in both experimental animal models and in clinical trials. The cost of production, continuous need to administer the antibody and side effects are limitations of monoclonal antibody therapy. The side effects attributed to adjuvants and autoreactive T cells, microhemorrages, aseptic meningioencephalitis, vasogenic edema are described in the review above mentioned [Hung et al., 2017].

Herein, all relevant art and publications in the field of AD to understand the research done so far in this area have been reviewed—(a.) the disease and how it progresses, (b.) how the disease is identified, (c.) identification of lead drugs, (d.) clinical trials and (e.) the existing technologies employed for drug delivery in various clinical trials. It is understood that currently there is no cure for AD which can be prescribed to an AD patient. Virtually all current strategies employed by practitioners include: (a.) imaging the brain for AD markers like plaques, tangles and other deposits; (b.) biochemical testing of blood samples or cerebrospinal fluid for confirmation; (c.) advising a control diet to the patient; and (d.) prescribing a drug that can slow the progression of the disease. The drug is initially prescribed for a particular dose and the dose is later increased when the disease is found to aggravate. At extreme case palliative care is advised. We have also understood that among the millions of elderly people that suffer from the disease world over, most of the patients are taken care at home and their care is a mammoth psychological and financial challenge to their caretakers. The above-mentioned facts indicate that there are some drugs available in the market that can slow the progression of AD. These drugs are neither stage specific nor stop or reverse the damage done by AD. These indicate the immense need to identify AD therapies that ought to be non-toxic, stage-specific and should revert the damage done by AD. The drugs should also be inexpensive.

Upon evaluating all the different causes that could be reasons for inciting and aggravating AD, a prime reason we found for aggravation of AD was ‘deposition of toxic amyloid forms without its clearance’. In the human body mechanisms adopted by normal brain to exclude Abeta includes (a.) activation of low-density lipoprotein receptor related protein (LRP-1) through hepatic: Herein the efflux of Abeta across the blood—brain barrier (BBB) is mediated by low-density lipoprotein receptor-related peptide 1 (LRP1) which allows Abeta to move into cerebrospinal fluid via perivascular or glymphatic pathways. Thereafter Abeta is reabsorped from cerebrospinal fluid (CSF) into the venous blood via arachnoid villi and blood-CSF barrier, or into the lymphatic system from the perivascular and perineural spaces and possibly via meningeal lymphatic vessels [Tarasoff-Conway J M, Carare R O, Osorio R S, Glodzik L, Butler T, Fieremans E, et al. 2015. Clearance systems in the brain-implications for Alzheimer disease. Nat Rev Neurol. 11: 457-470]; (b.) microglial activation: microglia when activated express some classes of receptors like class A scavenger receptor and CD36 that uptakes Abeta and delays AD progression. These cells also phagocyte Abeta and restrict Abeta accumulation in the brain. A class of glial cells like astrocytes secrete proteolytic enzymes that degrade Abeta, such as insulin-degrading enzyme, neprilysin, matrix metalloproteinase-9 and plasminogen that facilitate amyloid clearance.

Blood brain barrier (BBB): The blood brain barrier is a highly selective, regulated and efficient barrier or a tissue component that protects the brain from noxious molecules and pathogens. It has a surface area of 20 m² and is comprised of specialized endothelial cells strongly attached together via multiple binding proteins like occludins, claudins and junctional adherin molecules to form tight junctions and adherin junctions. The entry of molecules via BBB is controlled by an interface separated by brain endothelial cells on the blood side and supportive cells like astrocytes and pericytes on the brain side. The movement of necessary nutrients, signalling molecules and immune cells is regulated by influx and efflux at the endothelial junctions. Though it appears that endothelial forms a stringent barrier, these cells communicate with other cells like astrocytes, pericyte, microglia and neurons. The endothelium, pericytes, astrocytes, microglia and neurons thus form the neurovascular unit (NVU). The importance of some of these cell types are as mentioned: (a.) the pericytes of NVU have the ability to differentiate into multiple cell types, and therefore serve as a reservoir of multipotent stem cells in the brain; (b.) astrocytes are the most abundant cell type in the brain. The endfeet of these cells surround the endothelium of blood vessels which facilitate the phenotypic specialization of both cell types as well as their cross-talk. Astrocytes enhance expression of BBB transporters in brain endothelial cells and also release more neprilysin in response to Abeta-ApoE complexes. Most of these functions get affected during AD as astrocyte functionality shifts in the presence of high Abeta load resulting in astrogliosis, oxidative stress, and impaired glutamate; and (c.) microglia are cell types that constantly survey the CNS. These cells undergo transition to an activated phenotype on contact with an immune stimulus and secrete cytokines and vasoactive substances. These also physically shield blood vessels after injury. During AD, amyloid load causes excessive proinflammatory molecule secretion by microglia that causes direct or indirect neurotoxicity. The BBB may contribute to the indirect neurotoxicity via disruption, secretions, and/or aberrant transport mechanism. Apart from AD, disruption of BBB has been observed in diseases like type II diabetes, multiple sclerosis and even in stroke patients. Dysfunction or disruption of BBB causes leakage of circulating substances into the CNS that can be toxic; inadequate nutrient supply, buildup of toxic substances in the CNS, and increased entry of compounds that are normally extruded; as well as altered protein expression and secretions by endothelial cells and other cell types of the neurovascular unit that can result in inflammatory activation, oxidative stress, and neuronal damage [Erickson M A, Banks W A. 2013. Blood brain barrier dysfunction as a cause and consequence of Alzheimer's disease. J Cereb Blood Flow Metab. 33: 1500-1513]. Thus, maintenance of BBB is critical to reversing the pathogenesis of a disease. In this regard, new therapeutic molecules need to be identified that ameliorate BBB breakdown to serve as new therapeutic agents for patients suffering from AD, type 2 diabetes, stroke, seizures, meningitis, encephalitis, primary brain tumors, brain metastasis, brain abscesses, hemorrhagic stroke, septic encephalopathy, HIV-induced dementia and multiple sclerosis.

It will be seen in the next sections that a component that is produced by the human body and takes part in body activities like bone metabolism is taken into consideration to test its application against AD. It is hoped that the invention here can contribute to the campaign against AD.

### SUMMARY OF INVENTION

In an aspect of the present disclosure, there is provided a composition comprising carboxylated osteocalcin as represented by SEQ ID NO: 1, for use in treatment of abnormal amyloid deposition. Carboxylated osteocalcin or Gla-OC is a vitamin K dependent protein that is synthesized mainly by osteoblasts. This 49 amino acid long matured Gla-OC is carboxylated at 3 glutamate residues that enables the peptide to bind to calcium and hydroxyapatite.

In another aspect of the present disclosure, there is provided a method of treating a subject having disease involving abnormal amyloid deposits, said method comprising, administering to the subject a therapeutically effective amount of a composition comprising carboxylated osteocalcin as represented by SEQ ID NO: 1.

In yet another aspect of the present disclosure, there is provided a composition comprising carboxylated osteocalcin as represented by SEQ ID NO: 1, for use in reducing cleavage and phosphorylation of Tau protein in brain.

In a further aspect of the present disclosure, there is provided a composition comprising carboxylated osteocalcin as represented by SEQ ID NO: 1, for use in protection of a tissue component in an amyloid diseased mammal, wherein the tissue component is blood brain barrier comprising endothelial cells.

In one another aspect of the present disclosure, there is provided a composition comprising carboxylated osteocalcin as represented by SEQ ID NO: 1, for use in increasing level of undercarboxylated osteocalcin in circulation.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF ACCOMPANYING DRAWINGS

The following drawings form a part of the present specification and are included to further illustrate aspects of the present disclosure. The disclosure may be better understood by reference to the drawings in combination with the detailed description of the specific embodiments presented herein.

FIG. 1 (A) is a bar graph depicting the changes in the mRNA level of total osteocalcin upon Gla-OC treatment in 5×FAD amyloid over expressing transgenic mice (referred as Tg) as evidenced by Real Time PCR. (B.) is bar graph summarizing the changes in Gla-OC in serum of Wt and Tg animals administered vehicle or Gla-OC (300 ng per mouse per day) as evidenced by ELISA. (C.) is a bar graph summarizing the changes in Glu-OC (undercarboxylated osteocalcin) in serum of Wt and Tg animals administered vehicle or Gla-OC (carboxylated osteocalcin) as evidenced by ELISA. Data represents ±SD; n=5 mice per group. ^{#}P<0.05 versus Wt, *P<0.05 versus Tg in accordance with an embodiment of the present disclosure.

FIG. 2 illustrates the effect of carboxylated osteocalcin or Gla-OC in reducing amyloid load in amyloid over expressing brain: (A.) is bar graph summarizing the changes in soluble amyloid Aβ42 level in brain homogenates of wild type (Wt) and Tg mice when different concentrations of Gla-OC (subcutaneously per day) were administered as evidenced by ELISA. (B-C.) are bar graphs summarizing the changes in soluble and insoluble amyloid Aβ42 level in brain homogenates of untreated and Gla-OC (300 ng per mouse per day) Tg mice as evidenced by ELISA. (D-D′.) are representative microphotographs and respective bar diagram showing the changes in the number of specific Aβ42 plaque deposits in brains of Tg mice treated with and without Gla-OC as evidenced by immunohistochemistry. (E-E′.) Representative microphotographs and respective bar diagram showing the changes in the number of ThS⁺ fibrillar Aβ42 plaque deposits in brain of Tg mice treated with and without Gla-OC as evidenced by immunohistochemistry. (F.) is a bar diagram showing comparable differences in Aβ42 level in plasma of Tg animals treated with and without Gla-OC. (G.) is an illustration of swim traces of Morris Water Maze Test on probe trial given 1 hour after 5 acquisition trials demonstrating the rational well-being of the Gla-OC treated mice upon amyloid clearance. (G′.) The line graph compares the latencies of different groups to the target platform. Data represents ±SD, n≥8 mice per group. ^{#}P<0.05 versus Wt, *P<0.05 versus Tg in accordance with an embodiment of the present disclosure.

FIG. 3: (A.) Bar diagram showing changes in the mRNA level of specific genes involved in amyloid clearance as evidenced by qPCR. (B, B′.) Representative microphotographs and bar diagram depicting the changes in the number of GFAP⁺ astrocytes in brains of Tg mice with and without Gla-OC treatment as evidenced by immunohistochemistry. (C.) Representative microphotographs showing changes in protein expression of neprilysin in brains of Tg mice with and without Gla-OC treatment as evidenced by immunohistochemistry. Data represents ±SD, n≥8 mice per group. *P<0.05 versus Tg in accordance with an embodiment of the present disclosure.

FIG. 4 demonstrates cell sorting of astrocytes from 5×FAD Tg brain by flow cytometry. (A.) (a.) Forward/side scatter of dissociated frontal brain cortical cells maintained in cell culture. LIVE cells were gated using LIVE/DEAD yellow dye to select nuclei containing singlets. Thereafter, astrocytes and contaminating microglia were segregated as two distinct populations viz. EAAT1⁺CD11b⁻ and EAAT1⁻CD11b⁺ cells. (b-c.) EAAT1⁺CD11b⁻ cell fraction collected was cultured in specific culture media for 4 days. (b.) shows cells which is negative for EAAT1 (microglia), and (c.) shows cells positive for EAAT1 (astrocytes). (d.) Representative immunoblot of GFAP in cell lysates of EAAT1⁺CD11b⁻ sorted and cultured astrocytes. (B.) F-actin staining of sorted astrocytes exposed to Aβ42 peptide in the presence and absence of Gla-OC. Representative images by confocal microscopy are shown. (C.) Changes in Aβ42 uptake in astrocytes isolated from 5×FAD Tg mice in the presence and absence of Gla-OC. Figures are line graphs that shows changes in concentration of Aβ42 in cell culture supernatant and cell pellet as evidenced by ELISA. (D.) Representative images of Aβ42 immunoblot showing the size of intracellular Aβ42 aggregates at different time periods when exposed to Gla-OC and Aβ42. Culture supernatant (lyophilized) and pellets of cell cultures were lysed, separated by Tricine gel electrophoresis and changes in Aβ42 in supernatant and pellet were analyzed by immunoblot. (E.) Changes in intracellular LDH in astrocyte cell cultures (isolated from Wt and Tg mice) when exposed to Gla-OC and Aβ42 at different time periods. (F.) Representative immuno dot blot image of A11. Cell lysates from Gla-OC and Aβ42 treated cell cultures were spotted to nitrocellulose and probed using anti-A11 antibody (G.) Changes in protein expression of LRP-1 in astrocytes by when exposed to Aβ42 and Gla-OC. (a.) LIVE cells gated using LIVE/DEAD yellow dye. (b-b′.) Cell surface expression of LRP1 was determined by flow cytometry. (c.) Representative confocal microscopy images of astrocytes showing changes in cell surface expression of LRP-1. Data represents ±SD; n=3 independent experiments. *P<0.05 versus Aβ42, in accordance with an embodiment of the present disclosure.

FIG. 5 shows how Gla-OC stimulates catabolism of Aβ42 in primary astrocytes. (A.) Representative immunoblot showing induction of LC3II, a marker of autophagy in astrocyte cell cultures treated with Gla-OC and Aβ42. (B.) Gla-OC increases the size and number of acidic vesicles in astrocytes. (a.) Representative confocal images showing changes in the intracellular localization of HiLyte Aβ42 and Lysotracker Red (50 nM) with and without Gla-OC treatment. Mander's overlap co-efficient was calculated using Image correlation analysis, Image J (NIH). (b-c) Bar diagram shows the size and number of Lysotrackter puncta. Data represents ±SD; n=3 independent experiments. *P<0.05 versus Aβ42, in accordance with an embodiment of the present disclosure.

FIG. 6: Changes in lysosomal pH in astrocytes upon Aβ42 and Gla-OC treatments as determined by flow cytometry analyses of Lysosensor DND-189 stained cells. Data represents ±SD; n=3 independent experiments.

FIG. 7 Gla-OC induces nuclear translocation of TFEB in Aβ42 treated astrocytes. A. (a.) Representative confocal microscopy images shows the cellular status of transcription factor EB (TFEB) in astrocyte cell culture with Aβ42 and Gla-OC treatment. (a′.) Bar diagram showing TFEB (%) localized in nucleus and cytosol of astrocytes treated Aβ42 and Gla-OC+Aβ42. (b.) Representative agarose gel showing changes in Tfeb expression with Gla-OC and Aβ42 treatment under conditions when autophagy is inhibited as evidenced by RT-PCR. (c.) Changes in the mRNA level of Tfeb with Gla-OC and Aβ42 treatment under conditions when autophagy is inhibited as evidenced by q-PCR. (d.) Representative immunoblot showing changes in the protein expression of TFEB with Gla-OC and Aβ42 treatments. (e.) Representative immunoblot showing changes in the phosphorylation status of TFEB with Gla-OC and Aβ42 treatments. TFEB was immunoprecipitated from cell lysates of Aβ42 and Gla-OC+Aβ42 treated cells and probed with phospho-tyrosine antibody (B.) Changes in lysosomal markers in Aβ42 treated astrocytes in presence and absence of Gla-OC. (a-b.) Representative immunoblots showing changes in protein expression of LAMP2 and cathepsinD when exposed to Aβ42 in the presence and absence of Gla-OC. (c.) Bar diagram showing changes in the enzyme activity of cathepsinD when exposed to Aβ42 in the presence and absence of Gla-OC as evidenced by colorimetric assay, in accordance with an embodiment of the present disclosure.

FIG. 8 illustrates the ability of Gla-OC to modulate Aβ42 aggregation. (A.) Aβ42 aggregation in 1×DPBS (pH 7.4) at 37° C. as monitored with thioflavin T (ThT) binding in the presence of high, low and equimolar concentration of Aβ42. Aβ42 was used at 25 μM concentration and was constant for experimental groups. ThT fluorescence was estimated on aliquots of aggregation mixture isolated on day 3. (B.) Changes in ThT fluorescence when amyloid aggregates isolated from 5 month old 5×FAD Tg mice were exposed to Gla-OC. (C.) ThT binding with mutant Aβ42 (Tottori Japanese mutation—SEQ ID NO: 2) in the presence of Gla-OC. (D.) Transmission electron microscopy (TEM) of Aβ42 peptides exposed to Gla-OC. (a.) Aβ42 aggregated in 1×PBS (pH7.4) for 3 days; (b-d.) Aβ42 aggregates in the presence of Gla-OC (b.) equimolar ratio of Aβ42 (25 μM) and Gla-OC (25 μM), (c.) Aβ42 (25 μM)+10 ng/ml Gla-OC, (d.) Aβ42 (25 μM)+3 ng/ml Gla-OC, (e.) mutant Aβ42 (25 μM) aggregated for 3 days, (f.) mutant Aβ42 (25 μM)+Gla-OC, (g.) Amyloid aggregates from 5×FAD Tg brain, (h.) Amyloid aggregates from Tg brain when treated with Gla-OC for 3 days. (G.) (a.) HiLyte Aβ42 (10 μM) aggregated for 3 days, (b.) HiLyte Aβ42 (10 μM) exposed to Gla-OC (3 ng/ml) and aggregated for 3 days. All aggregation experiments were done in 1×DPBS (pH 7.4) at 37° C. All experiments were done in triplicates and representative images are shown in figure. (H.) ELISA data showing interaction of Aβ42 peptide and Gla-OC peptide. (I.) Bar diagram showing the different LDH activity in C8D1A astrocyte cell cultures when incubated with Aβ42 and Gla-OC aggregates for 24 hours, in accordance with an embodiment of the present disclosure.

FIG. 9 demonstrates how Gla-OC in modulates the Tau phosphorylation, cleavage and protects the blood brain barrier in db/db (A.) is an immunoblot representation showing the inhibitory effect of Gla-OC on the phosphorylation status of Tau5 protein in hippocampus of db/db mice. (B.) Representative immunoblot image illustrating the modulatory effect of Gla-OC on Tau cleavage in hippocampus of db/db mice. (C.) is a bar diagram depicting changes in mRNA level of insulin like growth factor-1 (IGF-1) in the liver and brain tissues of db/db mice treated with or without Gla-OC. (D.) is a bar diagram showing changes in protein levels of IGF-1 and insulin like binding proteins-3 (IGFBP-3) in serum of db/db mice after Gla-OC treatment. (E.) Bar diagram showing Evans Blue Dye in brain homogenate of db/db brains which is indicative of the extent of breach in blood brain barrier as evidenced by Absorbance measurement. The effect of Gla-OC in decreasing the dye level is also shown (F.) Representative immunoblot data showing changes in protein expression of occludin in vessel fraction of db/db treated with or without Gla-OC. Data represents ±SD; n=3 independent experiments. ^{#}P<0.05 db/db versus Wt, *P<0.05 db/db+Gla-OC versus db/db, ^{$}P<0.05 Wt+Gla-OC versus db/db+Gla-OC, in accordance with an embodiment of the present disclosure.

FIG. 10 demonstrates the effect of Amyloid β42 and Gla-OC on osteoblasts. MC3T3E1 osteoblasts differentiated in β-glycerophosphate (10 mM) and ascorbic acid (50 μg/ml) were pre-treated with Gla-OC (3 ng/ml) for 4 hours and then exposed to Amyloid β42 (under low serum conditions, 3% fetal bovine serum for 36 h). (A.) Representative immunoblot showing changes in the protein expression of osteocalcin. (B.) Representative agarose gel showing changes in the gene expression of Bglap2 gene (that encodes osteocalcin in mouse). (C.) qPCR data that shows changes in the mRNA level of Bglap2 gene under different conditions. Data represents ±SD; n=3 independent experiments. *P<0.05 versus basal, **P<0.05 versus Aβ42, in accordance with an embodiment of the present disclosure.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

Carboxylated osteocalcin (Gla-OC) is the carboxylated form of osteocalcin. It is also known as bone gamma carboxyglutamic acid containing protein (BGLAP) encoded by Bglap gene. The peptide is 49 amino acids in length and has 3 post translational carboxylation at glutamic acid residues in positions 17, 21 and 24 of the peptide. Carboxylation of osteocalcin is performed by carboxylase that is dependent on vitamin K. Carboxylation renders the peptide an alpha-helical structure and ability to bind calcium ions. Osteocalcin thus has high affinity for hydroxyapatite and hence found in high amounts in bone extracellular matrix. Initially it was presumed that Gla-OC plays role in mineralization but this aspect is under debate since knockout model for osteocalcin does not exhibit any impairment in bone remodeling or mineralization. The undercarboxylated form of osteocalcin has been shown to possess many physiological functions. However, till date, the actual role of Gla-OC is still ambiguous. Throughout the present disclosure, the term carboxylated osteocalcin and Gla-OC have been used interchangeably.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

The present disclosure provides the remedy for managing the amyloid related disorders in the form of composition comprising osteocalcin and a complex comprising Abeta 1-42 and undercarboxylated osteocalcin.

Sequence Listing

SEQ ID NO: 1 represents the amino acid sequence of
carboxylated osteocalcin (Gla = γ-Carboxyglutamic
Acid; Disulfide bridge: 23-29) Molecular weight
5929.5
YLYQWLGAPVPYPDPL-Gla-PRR-Gla-VC-Gla-LNPDCDELADHIGF
QEAYRRFYGPV
SEQ ID NO: 2 represents amino acid sequence for
Abeta42 with Tottori mutation DAEFRHNSGYEVHHQKLVFF
AEDVGSNKGAIIGLMVGGVVIA

The present disclosure is not to be limited in scope by the specific embodiments described herein, which are intended for the purposes of exemplification only. Functionally-equivalent products, compositions, and methods are clearly within the scope of the disclosure, as described herein.

In an embodiment of the present disclosure, there is provided a composition comprising carboxylated osteocalcin as represented by SEQ ID NO: 1, for use in treatment of abnormal amyloid deposits.

In an embodiment of the present disclosure, there is provided a composition comprising carboxylated osteocalcin as represented by SEQ ID NO: 1, for use in treatment of abnormal amyloid deposits, wherein treatment of abnormal amyloid deposits leads to treatment of diseases selected from the group consisting of type 2 diabetes mellitus, AL amyloidosis, secondary amyloidosis, familial amyloidosis, Alzheimer's disease, Down's syndrome, Idiopathic dialated cardiomyopathy, arthritis, tuberculosis, Lewy body variant of Alzheimer's, Parkinson dementia of Guam, spondylitis, Cerebral Amyloid Angiopathy (CAA) or congophilic angiopathy, Amyloidosis Dutch type, senile amyloid angiopathy, certain types of Creutzfeldt Jacob Disease, Kuru, fronto-temporal dementia with Parkinsonism linked to chromosome 17 (FTDP-17) caused by tau mutations, chronic traumatic encephalopathy, traumatic brain injury, Pick disease, corticobasal degeneration, dementia pugilistica and progressive supranuclear palsy. In one of the preferred embodiment of the present disclosure, the disease is Alzheimer's disease.

In an embodiment of the present disclosure, there is provided a composition comprising carboxylated osteocalcin as described herein, wherein the carboxylated osteocalcin leads to treatment of disease caused due to misfolded proteins. In another embodiment of the present disclosure, the misfolded protein is selected from the group consisting of Abeta42, alpha-synuclein, prion, tau protein, transthyretin and insulin.

In an embodiment of the present disclosure, there is provided a composition comprising carboxylated osteocalcin as described herein, wherein the carboxylated osteocalcin leads to clearance of abnormal amyloid deposits in brain.

In an embodiment of the present disclosure, there is provided a composition comprising carboxylated osteocalcin as described herein, wherein the carboxylated osteocalcin reduces plasma Abeta42 level.

In an embodiment of the present disclosure, there is provided a composition comprising carboxylated osteocalcin as described herein, wherein the carboxylated osteocalcin leads to treatment of cognitive disorder.

In an embodiment of the present disclosure, there is provided a composition comprising carboxylated osteocalcin as represented by SEQ ID NO: 1, for use in treatment of abnormal amyloid deposits, wherein the carboxylated osteocalcin is having fully carboxylated glutamic acid residues at positions 17, 21, and 24, and the osteocalcin is in calcium bound form.

In an embodiment of the present disclosure, there is provided a composition comprising carboxylated osteocalcin as represented by SEQ ID NO: 1, for use in treatment of abnormal amyloid deposits, wherein the carboxylated osteocalcin increases the activity of glial cells, thereby leading to treatment of abnormal amyloid deposits.

In an embodiment of the present disclosure, there is provided a composition comprising carboxylated osteocalcin as represented by SEQ ID NO: 1, for use in treatment of abnormal amyloid deposits, wherein the carboxylated osteocalcin increases the expression of neprilysin in glial cells and LRP-1 (low density lipoprotein like receptor-1) in brain, thereby leading to treatment of abnormal amyloid deposits.

In an embodiment of the present disclosure, there is provided a composition comprising carboxylated osteocalcin as represented by SEQ ID NO: 1, for use in protection of a tissue component in an amyloid diseased mammal, wherein the tissue component is blood brain barrier comprising endothelial cells.

In an embodiment of the present disclosure, there is provided a composition comprising carboxylated osteocalcin as represented by SEQ ID NO: 1, for use in protection of a tissue component in an amyloid diseased mammal, wherein the tissue component is blood brain barrier comprising endothelial cells, and wherein the composition increases expression of IGF1 and IGF-1 binding proteins in circulation and reinstates the expression of tight junction protein, thereby maintaining the integrity of blood-brain barrier.

In an embodiment of the present disclosure, there is provided a composition comprising carboxylated osteocalcin as represented by SEQ ID NO: 1, for use in reducing cleavage and phosphorylation of abnormal Tau protein in brain.

In an embodiment of the present disclosure, there is provided a composition comprising carboxylated osteocalcin as represented by SEQ ID NO: 1, for use in increasing expression of undercarboxylated osteocalcin in circulation.

In an embodiment of the present disclosure, there is provided a composition comprising: (a) carboxylated osteocalcin as represented by SEQ ID NO: 1, and (b) at least one pharmaceutically acceptable excipient or carrier.

In an embodiment of the present disclosure, there is provided a composition comprising carboxylated osteocalcin herein, wherein the composition is used in preparation of medicament.

In an embodiment of the present disclosure, there is provided a method of treating a subject having disease involving abnormal amyloid deposits, said method comprising, administering to the subject a therapeutically effective amount of a composition comprising carboxylated osteocalcin as represented by SEQ ID NO: 1. In one of the embodiment of the present disclosure, the composition is administered parenterally.

In an embodiment of the present disclosure, there is provided a method of treating a subject having amyloid pathology, said method comprising, administering to the subject a therapeutically effective amount of a composition comprising carboxylated osteocalcin as represented by SEQ ID NO: 1. In one of the embodiment of the present disclosure, the composition is administered parenterally.

In an embodiment of the present disclosure, there is provided a method of treating a subject having disease involving abnormal amyloid deposits, said method comprising, administering to the subject a therapeutically effective amount of a composition comprising: (a) carboxylated osteocalcin as represented by SEQ ID NO: 1, and (b) at least one pharmaceutically acceptable excipient or carrier.

In an embodiment of the present disclosure, there is provided a method of treating a subject having amyloid pathology, said method comprising, administering to the subject a therapeutically effective amount of a composition comprising: (a) carboxylated osteocalcin as represented by SEQ ID NO: 1, and (b) at least one pharmaceutically acceptable excipient or carrier.

Although the subject matter has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present subject matter as defined.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may vary.

The subsequent paragraphs describe the claimed invention of the present disclosure by way of examples. Examples and data have been depicted using mouse models such as 5×FAD mice for abnormal amyloid formation and deposition and db/db mice model for Tau pathology. All the studies performed using said mice are approved by the Institutional Animal Ethics Committee.

### List of Primers Used in the Study:

TABLE 1

| | | | Primer Assay |
|---|---|---|---|
| | | | catalogue number |
| S. | | Gene | from Qiagen |
| No. | Name of the protein | Name | (SA Biosceinces) |
| 1. | Osteocalcin Bone gamma carboxy | Bglap | PPM04465F |
| | glutamate protein | | |
| 2. | Insulin like growth factor-1 | Igf1 | PPM03387F |
| | or IGF-1 | | |
| 3. | Neprilysin | Mmel1 | PPM30863A |
| 4. | Beta-actin | Actb | PPH00037G |
| 5. | CD36 | Cd36 | PPMMO4465F |
| 6. | Iba1 (ionized calcium binding | Aif1 | PPM03752A |
| | adapter protein1) | | |
| 7. | Glial fibrillary acidic protein | Gfap | PPM04716A |
| 8. | Presenilin1 | Psen1 | PPM06211A |
| 9. | BACE1 (beta-secretase1) | Bace1 | PPM26538A |
| 10. | BACE2 (beta-secretase2) | Bace2 | PPM30427A |
| 11. | ADAM17 | Adam17 | PPM05316F |
| 12. | ADAM10 | Adam10 | PPM24900A |
| 13. | Low density like lipoprotein1 | Lrp1 | PPM05653A |
| 14. | Insulin like degrading enzyme | Ide | PPM05515A |
| 15. | Matrix metalloproteinase2 | Mmp2 | PPM03642C |
| 16. | Matrix metalloproteinase9 | Mmp9 | PPM63661C |
| 17. | CathepsinB | Cstb | PPM03626F |
| 19. | CathepsinD | Cstd | PPM03622A |

### Example 1

### Examples Related to Carboxylated Osteocalcin in Brain Amyloid Overexpressing 5×FAD Tg Mice

Osteocalcin is produced in the body primarily by osteoblasts and is believed to play role in bone mineralization process. There are three vitamin K dependent carboxy glutamic acid residues in osteocalcin which are critical for maintaining protein structure and regulation of the bone mineral maturation. Osteocalcin can get deposited onto bone or released into circulation where it correlates with bone formation. Since osteocalcin is a by-product of the bone, any alteration in its level in circulation may have clinical implications.

Effect of carboxylated osteocalcin (Gla-OC) against brain amyloid pathology was tested in B6SJL-Tg [(APPSwFlLon,PSEN1*M146L*L286V) 6799Vas/Mmjax] also known was the 5×FAD Tg mice. This mouse model was procured from Jackson Laboratory, Bar Harbour, Me. These 5×FAD Tg mice overexpress mutant human APP(695) with the Swedish (K670N, M671L), Florida (I716V), and London (V717I) Familial Alzheimer's Disease (FAD) mutations along with human PS1 harboring two FAD mutations, M146L and L286V. Both the transgenes are regulated by the mouse Thy1 promoter to drive overexpression in the brain. The model recapitulate major features of brain amyloid pathology and is a useful model of Abeta-42 induced amyloid plaque formation.

Two-month old 5×FAD Tg mice, both male as well as female were used for the study. For the purposes of this study wild type mice is referred to as Wt and 5×FAD mice is referred to as Tg. All experimental procedures in were approved by the Institutional Animal Ethics Committee. For experimentation, the mice were divided in below groups for treatment:

1. Wild type mice given vehicle (lx PBS or phosphate buffered saline; pH 7.4).
2. 5×FAD Transgenic mice given vehicle.
3. 5×FAD Transgenic mice given carboxylated osteocalcin (Gla-OC) subcutaneously at doses (300 ng-1000 ng per mouse of weight ranging from 25-28 g for 1 month).
4. Wild type mice given Gla-OC.

The following parameters were tested in the treated mice.

Foremost the status of osteocalcin in long bones and serum of amyloid overexpressing 5×FAD Tg mice was evaluated. Thereafter, the effect of osteocalcin administration against amyloidosis in brain was tested.
1. The status of osteocalcin expression in bone was determined by evaluating the mRNA level of Bglap2 gene (gene that encodes osteocalcin in mouse) using quantitative PCR or qPCR. Long bones of mice like femur and tibia were harvested from mice after sacrifice. RNA was extracted from tissue samples using RNA extraction kit from Qiagen, Netherlands according to the manufacturer's instruction. cDNA synthesis was performed using cDNA synthesis kit from Qiagen. To perform qPCR, 10 ng cDNA (per well) was amplified using the Light Cycler 480 Syber Green I Master reagent (Roche Diagnostics, Indianapolis, Ind.) and primers (commercially purchased from SABiosciences, Qiagen) in the Light Cycler 480 (Roche Diagnostics, Switzerland) under following cycling conditions: 3 min at 95° C., 15 sec at 95° C., 20 sec at 60° C., 25 sec at 72° C. for 40 cycles. Following amplification, fold changes in gene expression versus β-actin (reference) analysis was determined using the 2^{ΔΔCT} (Livak) method.
The primer used for qPCR is mentioned in Table 1.
2. Carboxylated (Gla-OC) and undercarboxylated (Glu-OC) forms of osteocalcin in serum of mice was analyzed by ELISA. Gla-OC and Glu-OC ELISA kits procured from Takara (Takara Bio, Mountain View, Calif., USA) were used for performing the assays.

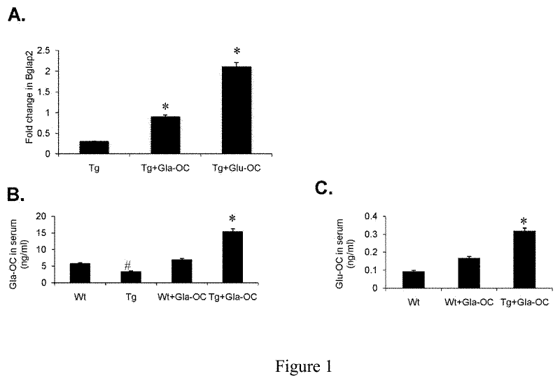

Results: It was observed that the long bones of 5×FAD transgenic mice demonstrated significantly lower level of Bglap2 mRNA level as compared to bones from wild type mice (FIG. 1A). This indicated that 5×FAD Tg animals suffer osteocalcin deficit. Also, the fasting level of Gla-OC in serum of 5×FAD Tg mice was lower than wild-type (FIG. 1B). Treatment with Gla-OC (300 ng per mouse of 25-28 kg body weight per day for 30 days) significantly (p<0.05) increased Bglap2 mRNA level (FIG. 1A). Gla-OC treatment significantly increased (p<0.05) the level of Gla-OC in 5×FAD Tg mice as compared to Tg controls (FIG. 1B). Treatment with Gla-OC also significantly increased (p<0.05) the level of Glu-OC in serum. Such significant rise in Gla-OC and Glu-OC was not seen in Wt mice treated with Gla-OC.

3. The levels of soluble and insoluble amyloid beta 42 in brain tissues were tested by ELISA. The soluble fraction refers to non-plaque associated Abeta. Brain homogenate at a concentration of 100 mg brain tissue per ml extraction reagent was prepared using 0.2% diaethylamine (DEA) and centrifuged at 100,000 g in an ultracentrifuge for 1 hour at 4° C. (54,000 rpm in ˜100.3 rotor). The supernatant (soluble fraction) was neutralized by addition of 1/10th volume of 0.5M Tris-HCl; pH 6.8 and vortexed gently. The insoluble fraction refers to plaque associated Abeta. For this 10% brain homogenate was prepared using RIPA (radioimmunoprecipitation assay) buffer containing protease-phosphatase cocktail (Sigma-Aldrich, St Louis, Mo., USA) was mixed with cold formic acid. To 200 microliter homogenate, 440 microliter of formic acid was added and mixed in a microcentrifuge tube and sonicated for 1 min on ice. The probe was moved up and down in between intervals. The homogenate was spun at 1,35,000 g for 1 hour which is approx. 50,000 rpm for 100.3 rotor. Further 210 microliter of supernatant was neutralized using FA neutralization buffer [1M Tris base, 0.5M Na₂HPO₄, 0.05% NaN₃, 60.57 g Tris base, 35.5 g Na₂HPO₄, 2.5 millilitre 10% NaN₃ were added and diluted to 500 ml and stored at room temperature since lower temperature will facilitate precipitation], and flash frozen. Prior to performing ELISA, samples were incubated for 5 min at 37° C. to clarify the solution and solubilize the precipitate. Total protein was estimated using Pierce BCA protein assay kit (all the steps were performed as per the manufacturer's protocol). ELISA of the samples were performed by specific kits procured from Thermo-Fischer (Invitrogen) and all the steps were performed as per the manufacturer's protocol.

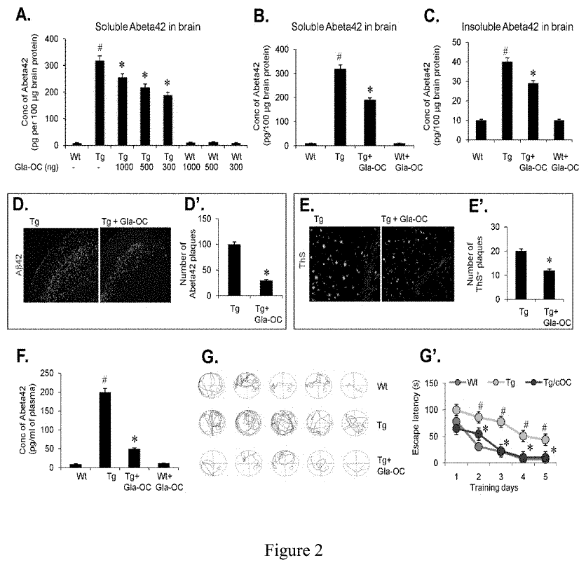

Results: The effect of Gla-OC on Abeta42 levels are depicted as FIGS. 2A-2C. 5×FAD Tg mouse brain displayed very high level of soluble and insoluble Abeta42. To determine the modulatory effect of Gla-OC on Abeta42 level in brain, different doses of Gla-OC from 300 ng to 1000 ng were tested and the level of soluble Abeta42 in brain was quantified. Though all the doses of Gla-OC tested for efficacy reduced the level of soluble Abeta42 in brain, the lowest concentration of Gla-OC studied (300 ng per mouse) showed maximum efficacy in reducing soluble Abeta42 in brain (FIG. 2A) and was hence was used for further experimentation. Gla-OC at 300 ng per mouse not only reduced the level of soluble Abeta42 in mouse brain but also significantly (p<0.05) reduced the level of insoluble Abeta42 (FIG. 2C). This experiment shows that Gla-OC has potency in reducing both plaque and non-plaque associated Abeta42 in brain.

For visual confirmation of the effect of Gla-OC on amyloid plaque reduction in brain, cryo sections brain were made as follows: Post sacrifice and whole animal perfusion fixation using 4% paraformaldehyde perfusion through the heart, the brains of the experimental animals were harvested and submerged in the same fixative for not more than 24 hours. Brains were then washed in 1×PBS or phosphate buffered saline (pH 7.4) and placed in 30% sucrose-PBS. The brains were then removed and washed in PBS. Brain samples were quick frozen in liquid nitrogen and then embedded in polyfreeze tissue freezing medium in a plastic mould inside a cryostat, wherein temperature was −20° C. The samples were then stored in −80° C. until sectioning. Sections of 15-20 micrometre was cut in a cryostat and laid over high adsorbent microslides. For unmasking antigens, slides were heated at 55° C. for 10 min followed by hydration in PBS for 5 min and then permeabilization using 0.1% Triton X-100 in PBS containing 1% BSA and 1% normal goat serum (IHC buffer). Brain sections were incubated in primary antibody (Anti-beta amyloid 1-42 antibody, AB5078P, Merk-Sigma-Aldrich) diluted in IHC buffer in a humidified chamber at room temperature overnight. After rinsing with PBS three times for 5 min each, sections were incubated with the appropriate secondary antibody conjugated with AlexaFluor 594 or 488 for 2 hours at room temperature. After rinsing with PBS, the coverslips were mounted with ProLong anti-fade mounting medium containing 4′,6-diamidino-2-phenylindole (DAPI) and imaged. For Thioflavin-S(ThS) staining, tissue slices were incubated in ThS (Sigma-Aldrich, St. Louis, Mo., USA) solution (0.025% in 50% ethanol) for 5-10 min. Amyloid burden quantification was performed by an investigator blind to the experimental groups.

Results: Quantitative image analyzes for Abeta42 in brain sections of 5×FAD Tg mice showed presence of both Abeta42⁺ plaque deposits (FIGS. 2D and D′) and ThS⁺ (thioflavinS) fibrillar deposits, a marker for fibrillary deposits of amyloid (FIGS. 2E and E′) in the cerebral cortex. Gla-OC treatment reduced Abeta42⁺ and ThS⁺ amyloid deposits in cortical region of 5×FAD Tg brain (FIG. 2D-D′ and 2E-E′, respectively). These assays showed that Gla-OC effectively reduced amyloid plaque deposits in cerebral cortex.

The above-mentioned observations are further supported by the graph represented in FIG. 2F, which depicts comparable difference in Abeta42 level in plasma of 5×FAD Tg animals and those treated with Gla-OC as measured by ELISA.

Result: 5×FAD Tg mice showed significantly elevated level of Abeta42 in serum. Treatment with Gla-OC significantly reduced (p<0.05) the level of Abeta42 in circulation with respect to transgenic control. It is evident that Gla-OC treatment effectively removes amyloid load from both brain as well as circulation.

Effect of Gla-OC treatment on cognitive function in 5×fad Tg mice: Morris water maze test or MWM is a navigation task performed to measure spatial memory, movement control and cognitive mapping. This experiment was conducted to test whether the reduction in Abeta amyloid load in brain of 5×FAD Tg mice reinstated the functionality of brain (which was otherwise disrupted owing to different factors, one significantly being amyloid overexpression). This was investigated via MWM. For the experiment, a tank with the diameter of 120 cm filled with the water having the temperature around 26° C. was divided into four quadrants, wherein one of the quadrants contained a transparent platform immersed, such that, the level of the water is about 1 cm above the surface of the platform. This platform is called the “hidden platform” since is it invisible to the mouse. On the first day of the training, the mouse is kept on the platform for the 15-20 seconds and then dropped in the opposite quadrant of the platform containing quadrant in the tank. The animal is allowed to search for the hidden platform and guided to the platform, if the animal is unable to find it in 120 seconds. The training was given for the five consecutive days, with the video recording and the track of the animal was also recorded using the Any-maze animal behaviour software. Time taken to find the hidden platform was calculated and plotted. The result of this experiment is depicted in FIGS. 1D&E.

Results: As can be observed in FIGS. 2G and 2G′, 5×FAD Tg mice suffered cognitive decline as evidenced by high escape latency or the time taken to reach the hidden platform inspite of the training. 5×FAD Tg mice treated with Gla-OC (300 ng) showed improvement in Morris water maze (MWM) test as evidenced by low escape latency. One can appreciate that, post treatment with Gla-OC, the 5×FAD Tg mice demonstrated similar profile as that of the Wt mice. It can therefore be concluded that, Gla-OC upon reducing amyloid load in 5×FAD Tg brain reinstates brain functionality.

Effect of Gla-OC in clearance of pathogenic amyloids in brain: To determine whether Gla-OC reduced amyloid pathology by promoting amyloid clearance, the expression status of Gfap and Aif1 genes that encodes glial fibrillary acidic protein (GFAP, a positive marker for astrocytes) and ionized calcium binding adapter molecule 1 (a positive marker for microglia) respectively were assessed by qPCR. Simultaneously, the expression status of those genes involved in amyloid uptake was also assessed.

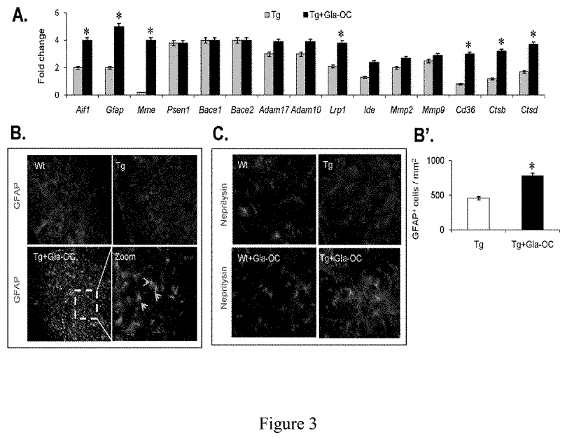

Results: qPCR data showed that the mRNA level of genes like Gfap, Aif1, Mme, Lrp1, Cd36, Ctsb and Ctsd were significantly increased (p<0.05) in brains of Gla-OC treated mice as compared to Tg control (FIG. 3A). These genes encode glial fibrillary acidic protein, allograft inflammatory factor 1 or ionized calcium binding adapter molecule 1 (otherwise known as Iba1), neprilysin, low density lipoprotein like receptor1, cluster differentiation 36, cathepsin B and cathepsin D respectively and are known to be unpregulated during amyloid clearance. 5×FAD Tg control mice showed low transcription of Mme, Cd36 and Cstb genes with respect to wild type.

Astrocytes internalize and degrade Abeta in brain. These cells are indispensable players in neural communication and recycle glutamate, regulate blood flow in central nervous system and immune response, release gliotransmitters and express ionotrophic and metabotropic neurotransmitter receptors. Neprilysin encoded by Mme gene is a predominant Abeta protease in astrocytes that cleaves Abeta42 and aids its clearance. Since genes like Gfap and Mme showed maximal expression with Gla-OC treatment in 5×FAD Tg mouse brain, the immunoreactivity of cerebral cortex towards GFAP and neprilysin antibody was assessed by immunohistochemistry (IHC). GFAP antibody used was monoclonal from e-Bioscience (Cat. 53989282) which was Alexa Fluor 488 conjugated. Neprilysin antibody used was sc-46656 (1:200 dilution) from Santa Cruz Biotechnology, Dallas, Tex., USA.

Result: FIG. 3B shows that immunoreactivity of GFAP was higher in cerebral cortex of Gla-OC treated 5×FAD Tg mice as compared to 5×FAD Tg. Gla-OC treatment significantly (p<0.05) increased the number of GFAP⁺ astrocytes in 5×FAD brain as compared 5×FAD Tg control (FIG. 3B′). The bar diagram depicts the total number of GFAP⁺ cells in a particular area analyzed by Image J (NIH, Bethesda). This shows that astrocytes play a role in Gla-OC induced pharmacological effect.

FIG. 3C shows that cerebral cortical regions of Gla-OC treated 5×FAD Tg mice showed significantly (p<0.05) higher immunoreactivity towards neprilysin as compared to Tg controls apparently because of the higher number of GFAP⁺ population in brain. This IHC data is in accord with the qPCR data and shows that Gla-OC treatment increases the population of astrocytes in cerebral cortex that also demonstrate an increased protein level of neprilysin, an Abeta degrading enzyme. Collectively the results demonstrate that protective effect of Gla-OC acts against amyloidosis or abnormal brain deposits involved astrocyte activation and degradation of Abeta pathogenic peptide.

### Example 2

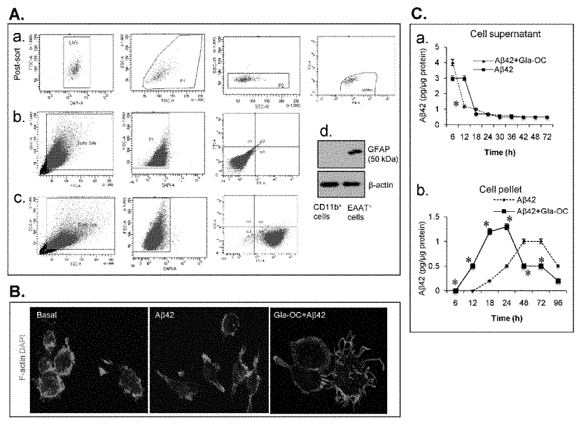

The process of how Gla-OC aids Aβ clearance by astrocytes in an amyloid overexpressing system was examined. Also, whether Gla-OC stimulates LRP1 expression was also investigated. For this, astrocytes were isolated from 2.5 month old Wt and 5×FAD Tg mice. After animal sacrifice and brain harvest, the myelin was carefully removed and the cerebral cortices were dissected and kept in ice-cold HBSS or Hank's balanced salt solution (without calcium and magnesium). The tissue was then subjected to enzymatic dissociation with papain enzyme at a final concentration of 8 U/ml in combination with DnaseI at a final concentration of 80 kunitz units per ml in PIPES [(piperazine-N N′-bis (ethanesulfonic acid) 1,4-piperazinediethanesulfonic acid)] based buffer with the addition of cysteine-HCl and ethylenediaminetetracetic acid (EDTA) in an incubator for 37° C. for 50 mins and then another 15 min after addition of extra 25 Kunitz units/ml DnaseI. The mix was spun at 200 g for 15 min and pellet was titurated to get a single cell suspension. The cells were resuspended in minimal essential media (MEM) with 1% bovine serum albumin or BSA and filtered through a 70 micrometre cell strainer (BD Bioscience). The cells were then layered over 90% Percoll gradient and centrifuged at 200 g for 15 min at 4° C. The top phase was discarded and Percoll layer containing cells and myelin layer was collected and diluted 5 times using MEM/1% BSA. This was spun at 200 g for 10 min at 4° C. The cells were then plated in poly D-Lysine coated and grown in Dulbecco's modified essential media (DMEM) supplemented with 10% fetal bovine serum and 1% antibiotics (Gibco). The media was changed every 2 days. After cells reached sufficient confluency, cells were dissociated using PBS containing 0.5% BSA and stained using EAAT1 antibody (Abcam, 1:100), CD11b (eBiosciences 1:200), Fc receptor block CD16/32 (1:200 BD Pharmigen) and immunoglobulin G isotype control (1:150, Thermo Scientific). The antibodies were incubated for 30 min at 4° C. and secondary antibodies like PE conjugated goat anti-mouse (1:25, eBiosciences), streptavidin APC-Cy7 (1:125, Biolegend) were added and incubated for 15 mins. The cells were washed in FACS staining buffer (eBiosciences) by spinning at 200 g for 5 mins and resuspended in buffer containing 8 g/L NaCl, 0.4 g/L KCl, 1.77 g/L Na₂HPO₄.2H₂O, 0.69 g/L NaH₂PO₄.2H₂O, 2 g/L D-glucose, pH 7.4 with 3% BSA and Yellow viability dye (Thermo Scientific). Using BD FACS Aria I 9BD Bioscience), EEAT1 positive cells were sorted on EAAT1⁺/CD11b⁻ expression and mmicroglia were separated on CD11b⁺ expression after gating the dead cells based on the viability dye used (FIG. 4Ab-c). The sorted cells were resuspended in DMEM/F-10 media (Thermo Scientific) with 1% antibiotics and further cultured.

Result: The integrity of the cells was checked by immunoblotting using GFAP antibody. EAAT1⁺/CD11b⁻ fraction showed positivity towards GFAP (FIG. 4Ad).

Astrocytes derived from 5×FAD Tg mice were pre-treated with Gla-OC (3 ng/ml) for 30 min and then exposed to oligomeric Abeta42 (1 micromolar) for 24 hours. The morphological changes of cells with treatment were assessed by phalloidin F-actin (Invitrogen). To quantitate whether Gla-OC stimulates the clearance of Abeta42 by astrocytes, the Abeta42 content of cell-culture supernatant and the cell lysate was determined by ELISA using specific Abeta42 detection kit from Invitrogen (FIG. 4C) and Western blotting analysis (FIG. 4D) using beta-amyloid D9A3A antibody from Cell Signaling Technology.

Results: Addition of Abeta42 (1 μM) to astrocytes from 5×FAD Tg mice did not show significant changes in cell morphology as evidenced by F-actin staining (FIG. 4B). Pre-treatment of cells with Gla-OC (3 ng/ml) followed by Abeta42 showed significant changes in astrocyte morphology. This showed that Gla-OC induced cellular changes within the astrocyte.

After addition of 1 μM of Abeta42 peptide into the medium, the Abeta in supernatant sharply declined at 18 h and then gradually decreased and dropped to minimum at 24 h after which no significant change in Abeta42 was noted in cell culture supernatant (FIG. 4Ca). When cell cultures were pre-treated with Gla-OC and then treated with Abeta42, the reduction in Abeta42 in cell culture supernatant was detected as early as 12 hours. This data indicated that presence of Gla-OC stimulated uptake of extracellular Abeta42 (FIG. 4Ca).

The aggregation of Abeta42 was detected in cell pellet by immunoblotting (FIG. 4D). Interestingly, most of intracellular Abeta42 in astrocytes are found to be oligomers (as the molecular weight of the brand is about 50 kDa) post 24 hours, suggesting that astrocytes can phagocytose oligomeric Abeta42. There were comparable differences in the protein level of Abeta42 oligomers in Abeta42 alone and Abeta42+Gla-OC treated cell cultures. Densitometric analysis using Image J (NIH) shows that the immunoreactivity towards oligomeric forms of Abeta42 was significantly reduced in Abeta42+Gla-OC cell lysates isolated at 48 and 96 hours (FIG. 4D).

Abeta42 oligomers are more toxic to cells as compared with aggregates such as Abeta fibrils and amyloid plaques. Since the internalized Abeta42 in astrocyte cell cultures were found to be oligomeric, the cytotoxic effect of internalized Abeta42 was evaluated in astrocytes isolated from both 5×FAD Tg and Wt mice by lactate dehydrogenase assay (LDH) kit (Sigma-Aldrich). The neurotoxicity of these oligomers were tested by dot blot analysis using conformation specific antibody A11 (Invitrogen). A11 antibody recognizes amino acid sequence-independent oligomers of proteins or peptides and not monomers or mature fibers of proteins or peptides. A11 is shown to recognize oligomeric species of several other amyloidogenic polypeptides including Abeta42, human insulin, prion, polyglutamine, lysozyme, alpha-synuclein and yeast prion Sup35.

Results: FIG. 4E shows that the intracellular level of LDH was significantly higher in transgenic astrocyte cultures as compared to wild-type. In both types of cell cultures, be it transgenic or wild-type derived astrocytes, Gla-OC treatment significantly (p<0.05) reduced intracellular level. A time dependent decrease in intracellular LDH was observed in transgenic astrocyte cultures exposed to Gla-OC.

Dot blot assay showed that the immunoreactivity of the cell pellets towards A11 antibody was lower in cell lysates from Abeta42 and Gla-OC treated cell cultures as compared to Abeta42 alone treated cell cultures (FIG. 4F).

LRP1 endocytic function plays a critical role in Abeta42 uptake and Abeta42 accumulation in lysosomes. Thus LRP-1 has role in Abeta42 metabolism. Since Lrp1 gene induction was evident in cell cultures treated with Gla-OC and Abeta42, the cell surface protein level of LRP-1 was assessed by flow cytometry and immunocytochemistry. For flow cytometry, cells after treatments with Abeta42 and Gla-OC for 24 hours were washed in 1×PBS (pH 7.4), detached by cell dissociation solution (Sigma-Aldrich), spun at 100 rpm for 5 min and resuspended in FACS (fluorescence activated cell sorting) buffer. The cells were blocked with Fc receptor block CD16/32 in ice for 10 min, washed with FACS staining buffer and exposed to Alexa fluor conjugated LRP-1 antibody (Abcam) for 20 mins at 4° C. Cells were washed again in FACS staining buffer, fixed using fixative (eBiosciences) and analyzed on BD Aria I (BD Bioscience). For immunocytochemistry, cells were grown in poly L-lysine coated coverslips (BD Bioscience), exposed to Abeta42 and Gla-OC for 24 hours. Cells were washed with 1×PBS (pH 7.4), fixed in 4% paraformaldehyde for 20 mins, washed with 1×PBS (pH 7.4) thrice, blocked with 3% goat serum (Gibco) for 20 min, washed with 1×PBS (pH 7.4) twice and incubated overnight with LRP-1 antibody (1:100, Santa cruz Biotechnology). Cells were washed thrice with 1×PBS (pH 7.4) thrice at anterval of 5 min and incubated with Alexa fluor conjugated goat anti-mouse IgG secondary antibody for 30 mins. Cells were again washed in PBS and mounted using DAPI containing mountant (Invitrogen) and view under confocal microscope (Ziess).

Result: Flow cytometry analysis showed that the percentage of cells positive for LRP-1 was higher in Gla-OC and Abeta42 treated cell cultures as compared to Abeta42 alone treated cell cultures (FIG. 4Gb-b′). This was further confirmed by surface staining of LRP1 and confocal microscopy (FIG. 4Gc). The results showed that Gla-OC promoted uptake of amyloid-beta in LRP-1 dependent mechanism. We next assessed how Abeta42 degradation occurs in Gla-OC exposed astrocytes. This was assessed by first evaluating the protein expression of LC3II by immunoblot analysis. Under normal conditions excess cargo in cell is cast-off by a cell through a recycling pathway called ‘autophagy’. The first step in autophagy is autophagosome formation wherein LC3I a cytosolic microtubule associated protein light chain 3 gets lipidated to form LC3II and recruits to autophagosomal membranes to allow cells to be LC3II*, a bona fide marker of autophagy and the first step towards autophagic lysosomal degradation. Another requirement for autophagosomes to be functionally relevant is downstream fusion with lysosomes. Under normal conditions, autophagosomes fuse with endosomes to form a higher order organelle called autophagosomes or ‘amphisomes’, which then matures to form ‘autolysosomes or terminal lysosomes’. The efficiency of autophagosome/lysosome fusion was evaluated using Lysotracker Red, a fluorescent dye that preferentially accumulates in vesicles with acidic pH. The cells were treated with Lysotracker Red for 20 min and fixed using ice-cold methanol. After confocal microscopy (Ziess) the images were analyzed using Image J (NIH). The co-occurrence of the two signals is shown as Mander's overlap co-efficient (MOC) in the range of values 0 to 1. For measurement of cellular pH, cells were exposed to LysoSensor Green DND-189 (pKa=˜5.2), another cell permeabilizing agent. This dye accumulates in acidic intracellular organelles and its fluorescence increases with acidic environments and decreases with that of alkaline. The status of lysosome biogenesis was assessed by immunoblot or immunocytochemistry. Lysosomal biogenesis is a collective term used to describe numerous events like sense nutrient availability, ‘lysosome to nucleus’ signaling cascade and energy metabolism. To evaluate lysosome biogenesis the status of LAMP2 (by immunoblot), cathepsins (by immunoblot and colorimetric activity based assay) and TFEB or transcription factor EB (by immunocytochemistry for cellular localization of transcription, by PCR for evaluating gene transcription, by immunoblot for protein expression and by co-immunoprecipitation and immunoblot to check phosphorylation of TFEB) were evaluated. Amongst these markers evaluated, LAMP2 or lysosomal-associated membrane protein 2 is a receptor that regulates fusion of the lysosome with the autophagosome and also degradation of specific cytosolic cargo during chaperone-mediated autophagy. Cathepsin D is an aspartic endoprotease that is ubiquitously distributed in lysosomes which degrades proteins and activates precursors of bioactive proteins in pre-lysosomal compartments. The antibody ab6313 (Abcam) used herein against cathepsin D recognizes three different bands in cell lysates corresponding to pre-cathepsin D (52 kDa), single chain of mature cathepsin D (48 kDa) and the double chain of cathepsin D (32 kDa). Transcription factor EB (TFEB) is a basic helix-loop-helix-zipper that interacts with innumerable lysosomal genes containing the CLEAR (Coordinated Lysosomal Expression and Regulation) motif for regulating lysosomal proliferation, expression of degradative enzymes, autophagy, lysosomal exocytosis and lysosomal proteostasis. Activation of this transcription factor is tightly regulated by its cellular localization. Under fed conditions, master growth regulator mTOR phosphorylates TFEB (inactive) and retains TFEB on lysosomal membrane, which refrains TFEB from migrating to nucleus. Upon stimulus, TFEB gets translocated to nucleus where it induces transcriptional activation.

LAMP2 and cathepsin D protein expression and activity were determined in lysosomal fraction isolated from cell cultures. Lysosomes were isolated using a lysosomal enrichment kit from Pierce Biotechnology according to the manufacturer's instructions. Briefly, the lysates were combined with OptiPrep to a final concentration of 15%, and placed on top of a discontinuous density gradient with the following steps from top to bottom: 17%, 20%, 23%, 27%, and 30%. After centrifugation for 2 h at 1,45,000 g, the top fraction containing the lysosomes was collected. Other membrane fractions present in the gradient were also collected and combined. The lysosomal fraction and the rest of the cellular membranes were diluted at least three times with PBS, and pelleted by centrifugation for 1 h at 18,000 g. The membranes were washed once with PBS and recovered by centrifugation at 18,000 g.

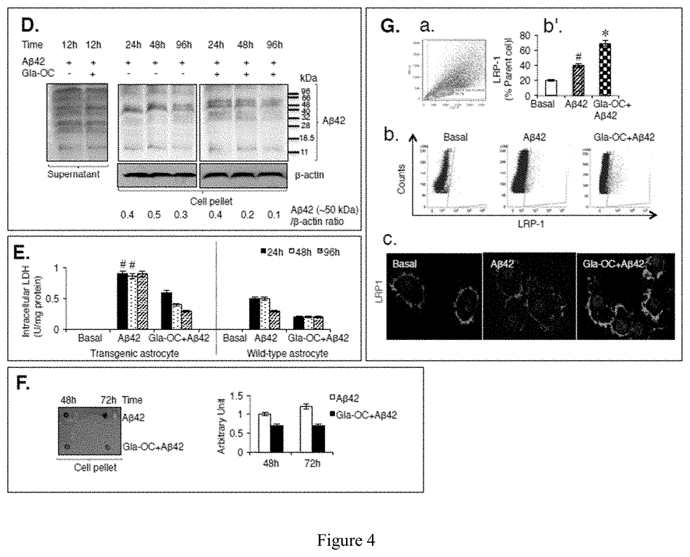

Results: Pre-treatment of astrocytes with Gla-OC promoted autophagy induction as evidenced by LC3II expression (FIG. 5A) and there was a comparable difference in the expression of LC3II between Gla-OC+Abeta42 and Abeta42 alone treated cells. The autophagy flux was also active in Gla-OC treated cells as evidenced by augmented accumulation of LC3II when exposed to lysomotrophic-basifying chloroquine (30 nM, 3 h, autophagy inhibitor) (Note: the cells were grown in autologous serum and so cells are not starved).

Pre-treatment with Gla-OC also improved co-localization of HiLyte Abeta42 with Lysotracker Red as compared to Abeta42 alone treated cells (FIG. 5Ba). Image J (NIH) analysis showed that the degree of co-localization of HiLyte Abeta42 with Lysotracker Red (evident as yellow puncta) was 0.633 in Gla-OC and HiLyte Abeta42 treated cultures while it was 0.412 in HiLyte Abeta42 alone treated cells (Higher MOC greater the degree of signal colocalization).

FIG. 5Bb shows the size of the Lysotracker Red puncta (acidic organelles) in astrocyte cultures exposed to Abeta42 alone and Gla-OC+Abeta42. It is evident that the sizes of the Lysotracker Red puncta was smaller in HiLyte Abeta42 treated cells and these were of sizes <0.25 μm². On the other hand, basal cell cultures and Gla-OC+HiLyte Abeta 42 treated cell cultures showed a similar size population (<0.25 to >1 μm²) of Lysotracker Red puncta. FIG. 5Bc shows the number of Lysotracker puncta in astrocyte cultures exposed to Abeta42 alone and Gla-OC+Abeta42. The number of the Lysotracker Red puncta was 4-fold lower in HiLyte Abeta42 treated cells as compared to basal. These puncta was also found mostly towards the periphery of the cell, which are likely to be less acidic. In Gla-OC+HiLyte Abeta42 treated cells, the number of Lysotracker puncta was higher and reaching basal values. Also, the acidic puncta was away from the periphery of the cell.

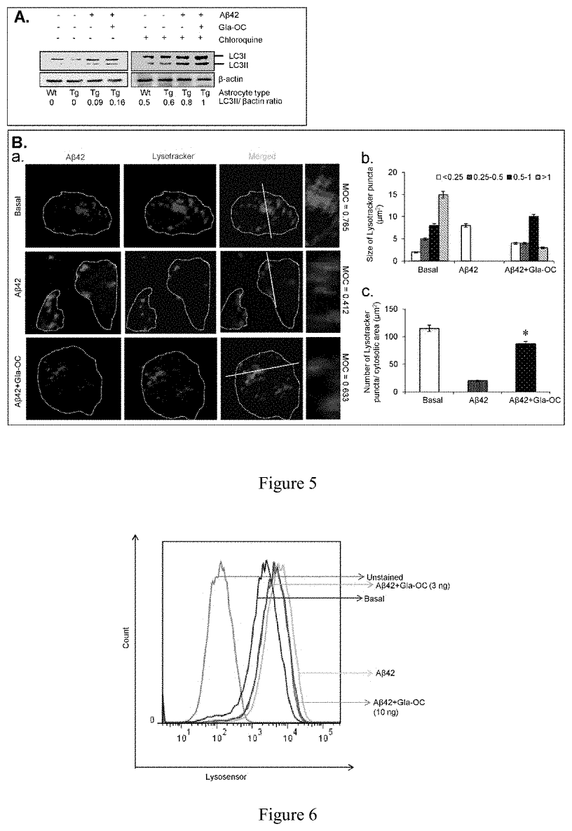

To determine the functionality of the acidic puncta, the pH of cell was determined. As can be seen in FIG. 6, astrocytes from transgenic mice exhibited a hike in lysosomal pH upon Abeta42 endocytosis, which was counteracted with Gla-OC treatment, interestingly in a concentration ‘independent’ manner. These results in tandem show that Gla-OC exerts a protective effect on maintaining the lysosomal size, numbers, position and pH and thus facilitates fusion of Abeta cargo with acidic organelles like autophagosomes and subsequent catabolism of endocytosed Abeta42.

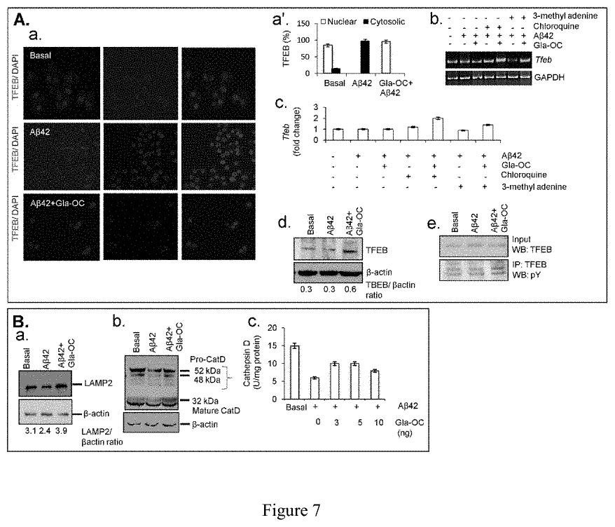

The status of master regulator of lysosome biogenesis was evaluated by determining its cellular location. Image analysis shows that basal astrocytes from transgenic mice show mostly nuclear localization of TFEB and comparatively less cytosolic localization (FIG. 7Aa-a′). When Abeta42 is added to cell cultures and subsequently allowed to endocytose, the cellular localization of TFEB is found completely cytosolic (FIG. 7Aa′). When cells were pre-treated with Gla-OC and then exposed to Abeta42, complete nuclear localization of TFEB was evident indicating that Gla-OC promotes nuclear translocation of TFEB. However, no change in the transcription of Tfeb gene was seen with addition of Abeta42, Gla-OC or both as evidenced by reverse transciptase PCR (FIG. 7Ab) and qPCR (FIG. 7Ac). The transcription of Tfeb gene however increased in the presence of autophagy inhibitors such as chloroquine and 3-methyl adenine (FIG. 7Ac). Immunoblot analysis showed that the protein level of TFEB was not altered with Abeta42 addition but an increase in the protein expression of TFEB protein was detected when astrocytes were pre-treated with Gla-OC (FIG. 7Ad). To determine the rationale for this change the phosphorylation of TFEB, an important post translational modification that determines its functionality was assessed. For that TFEB protein was immunoprecipitated from cell lysates using appropriate antibody and later probed with phosphotyrosine antibody. It is evident from FIG. 7Ae that presence of Gla-OC in Abeta42 treated cell cultures promoted higher phosphorylation of TFEB.

The functionality of TFEB was assessed by evaluating the protein expression of LAMP2 and cathepsin D, which are encoded by genes regulated by TFEB. FIG. 7Ba shows that LAMP2 expression in the isolated lysosomes is lower than in Abeta42 treated astrocyte cultures while it was significantly (p<0.05) increased above basal values in Gla-OC and Abeta42 treated astrocyte cultures. FIG. 7Bb shows decreased expression of pro-cathepsin D as well as reduced proteolytic cleavage to mature forms in Abeta42 treated cell cultures. Gla-OC treatment substantially improved the protein expressions of both pro-cathepsin D as well as mature cathepsin D. The result was confirmed by activity-based assay that showed that Gla-OC stimulated cathepsin D activity in a concentration dependent manner (FIG. 7Bc).

### Example 3

### Examples Related to the Ability of Gla-OC to Modulate Aβ42 Aggregation and Reduce its Toxicity

Protein aggregation and subsequent amyloid fibril formation is a common feature underlying a wide range of disorders like Alzheimer's disease, Parkinson's disease and type 2 diabetes. Thioflavin T (ThT) is a benzothiazole salt commonly used as a probe to monitor amyloid fibril formation in vitro. Upon binding to amyloid fibrils, ThT produces a strong fluorescence signal at approximately 482 nm when excited at 450 nm. This fluorescence enhancement upon binding to amyloid is attributed to the rotational immobilization of the central C—C bond connecting the benzothiazole and aniline rings. To determine the modulatory effect of Gla-OC on Abeta42 aggregation, Abeta42 peptide was brought to monomeric state. For this Abeta42 was dissolved in HFIP to a protein concentration of 500 μM. The samples in HFIP were left undisturbed for 30 min, and then HFIP was evaporated in a chemical hood overnight and then put under vacuum (Eppenforf) for 1 h to complete HFIP treatment. To make amyloid fibrils, HFIP-treated Abeta42 protein was dissolved in CG buffer (20 mM CAPS, 7 M guanidine hydrochloride, pH 11), and concentration was determined using absorbance at 280 nm and an extinction coefficient of 1.28 mM-1 cm-1. Thereafter, Abeta42 samples were diluted 20-fold into PBS buffer (50 mM phosphate, 140 mM NaCl, pH 7.4), and then incubated at 37° C. for 3 days to allow fibril formation. The progress of aggregation was monitored using thioflavin T in a fluorimeter (Fluoromax4, Horiba). The final concentration of Abeta42 in the aggregation reaction is 25 μM. The fibril concentrations were considered to be the same as the starting monomer concentration, with the notion of complete conversion from monomers to fibrils. To determine the modulatory effect of Gla-OC, the Abeta42 reaction mixture was incubated with Gla-OC at 3 ng/ml, 10 ng/ml and at 1:1 ratio. Gla-OC was added at 0 hour time point. Aliquots of reaction mixture were also visualized by transmission electron microscopy (TEM). For that 10 microliters of sample was applied to a 200-mesh carbon coated grid for 5 minutes, stained in 3% uranyl acetate for 5 minutes, rinsed, and air-dried. The grids were examined using a TEM microscope (Technai G220) at 80 kV. Alternately HiLyte Abeta42 was also incubated with Gla-OC and the resultant product was visualized under confocal microscope (Ziess).

For A11 dot blot assay, five microliters of each sample were spotted onto nitrocellulose membrane and allowed to air-dry. Tris-buffered saline (20 mM Tris, 0.8% NaCl, pH 7.4) containing 0.001% Tween-20 (TBST) was used for washing and dilution. The membrane was blocked for 1 hour with 5% BSA in TBST, washed 3×10 minutes, incubated for one hour in primary anti-A11 antibody (1:5,000 in 3% BSA/TBST), washed 3×10 minutes, incubated for 30 minutes in HRP-conjugated secondary antirabbit antibody (1:10,000 in 3% BSA/TBST), and washed 3×10 minutes. The membrane was developed using chemiluminescence reagents (Pierce) and imaged in LAS400 imager and analyzed using Image J (NIH).

For determining interaction of Gla-OC with Abeta42, ELISA was performed. For that Maxisorp ELISA plates (NUNC, Denmark) were coated overnight at 4° C., with 1-500 mg/ml of the relevant Gla-OC in 0.1 M carbonate buffer pH 9.6 and blocked 2 hours at room temperature with a blocking buffer containing 3% BSA/0.05% Tween 20 in phosphate buffered saline (PBS, 0.1 M phosphate buffer, 150 mM NaCl, pH 7.2). For the binding experiments Abeta42 was diluted at the preferred concentration (25-50 micromolar was tested and 25 micromolar chosen) in blocking buffer. For the competition experiments serial binary dilutions of the synthetic peptide and a blank sample were prepared in blocking buffer containing the desired antibody against Abeta (Novus) at the desired concentration. 100 μl of these solutions were added to the wells and incubated at room temperature 1-3 hours. The wells were washed 4 times with 1×PBS/0.05% Tween 20 and 100 μl of horseradish peroxidase conjugated secondary antibody (anti-rabbit) diluted 1:20,000 in blocking buffer were added in each well. After incubation for one hour at room temperature and subsequent washing, the bound horse radish peroxidase conjugate was detected by adding 100 μl of tetramethyl benzidine (TMB) substrate. The peroxidase reaction was stopped after 5 minutes by the addition of 50 μl 0.5 M H₂SO₄. Optical densities at 450 nm were measured using an ELISA reader (Tecan). The assay was performed in triplicates.

For LDH assay, aliquots of aggregation mixture was exposed to C8D1A astrocytes (ATCC) that was grown in DMEM high glucose supplemented with 10% fetal bovine serum. Briefly, cells were exposed to aggregation mixture in 2% fetal bovine serum condition (low serum) for 24 hours and the extracellular LDH activity was determined using LDH assay kit (Sigma-Aldrich).

For preparation of amyloid seeds 5×FAD Tg mouse brain (4 month old) the procedure reported by Stohr et al. (2012) published in PNAS (109: 11025-11030) was followed. Brains were homogenized in 1.5 ml calcium- and magnesium-free PBS and diluted 1:1 with 2×citrate lysis buffer (20 mM citrate, pH 6; 2% (wt/vol) Triton X-100; 274 mM NaCl; 2 mM EDTA) and incubated for 30 min on ice. The homogenate was thereafter adjusted to 18% (wt/vol) iodixanol with a 60% (wt/vol) iodixanol stock solution. Two identical step gradients of 35, 30, and 18% (wt/vol) (containing brain homogenate) were created and centrifuged in an ultracentrifuge at 60,000×g for 20 min (without the rotor brake activated). The top lipid layer was discarded; the next two layers as well as the interphases above and between them were collected and diluted 1:1 with citrate buffer (10 mM citrate buffer, pH 6; 137 mM NaCl; 1 mM EDTA). This sample was used as the top layer of a second gradient followed by two layers of 26 and 35% (wt/vol) iodixanol. This gradient was centrifuged at 60,000×g for 40 min (again without the brake). The top layer was discarded; the next two layers and interphases above and between them were collected and diluted with 2 mL of citrate buffer. This sample was split into 1-mL aliquots and centrifuged for 40 min at 21,000×g in siliconized microcentrifuge tubes (Midsci). The supernatant was discarded and each pellet resuspended in 400 μL Tris buffer (10 mM, pH 8.3) containing 1.71 M NaCl and 1% (wt/vol) zwittergent 3-14. After centrifugation for 30 min at 21,000×g, the supernatant was discarded, and the pellets were resuspended in 100 μL TMS buffer (50 mM Tris-HCl, pH 7.8; 100 mM NaCl, and 10 mM MgCl2) and combined into two tubes. Each sample was then treated with 300 units/mL benzonase (Novagen) for 2 h at 37° C., followed by centrifugation for 30 min at 21,000×g. To eliminate any residual protein contaminants, the resuspended pellets were treated with 40 μg/mL proteinase K (Fisher Scientific) for 1 h at 37° C., and the digestion was stopped by the addition of 2 mM PMSF. This digested sample was adjusted to 1.71 M NaCl and centrifuged for 30 min at 21,000×g through a 100 μL sucrose cushion (1 M sucrose). The pellet was resuspended in 0.1 M sodium acetate buffer and centrifuged for 30 min at 21,000×g. The final pellet was resuspended in 100 μL dH2O. For bioassays, purified samples were diluted 1:6 in freshly filtered PBS, snap frozen in liquid nitrogen, and stored at −80° C.

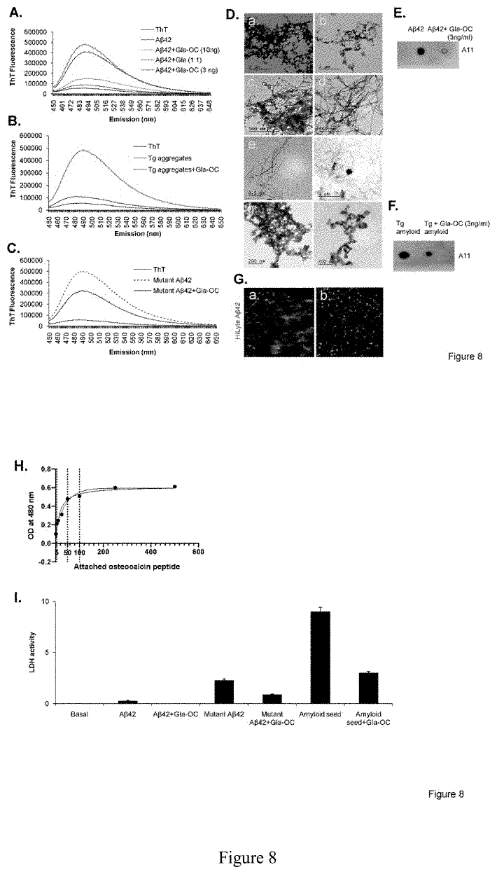

Results: FIG. 8 illustrates the ability of Gla-OC to modulate Aβ42 aggregation. FIG. 8A shows that significant modulatory effect on Abeta42 aggregation can be achieved with lower concentration of Gla-OC (3 ng/ml) as evidenced by reduced ThT fluorescence at 480 nm emission. An equimolar concentration of Abeta42 and Gla-OC does not have significant effect on Abeta42 aggregation. FIG. 8B shows that amyloid seeds isolated from brains of 5×FAD transgenic mice produces high ThT fluorescence which is counteracted when these seeds were pre-incubated with Gla-OC (3 ng/ml). The effect of Gla-OC (3 ng/ml) against mutant form of Abeta42 (25 micromolar; Tottori Japanese mutant; sequenceDAEFRHNSGYEVHHQKLVFFAEDVGSNKGAIIGLMVGGVVIA) was tested and found that Gla-OC inhibits aggregation of mutant Abeta42 as well (FIG. 8C). FIG. 8D is a visual confirmation of the amyloids treated with Gla-OC.

(a.) Aβ42 aggregated in 1×PBS (pH7.4) for 3 days—amyloid like; (b.) equimolar ratio of Aβ42 (25 μM) and Gla-OC (25 μM)-amyloid like; (c.) Aβ42 (25 PM)+10 ng/ml Gla-OC—fibril like; (d.) Aβ42 (25 μM)+3 ng/ml Gla-OC—mature fibril like; (e.) mutant Aβ42 (25 μM) aggregated for 3 days—mature fibril; (f.) mutant Aβ42 (25 μM)+Gla-OC—thinner and lighter fibril; (g.) Amyloid aggregates from 5×FAD Tg brain—thick amyloid; (h.) Amyloid aggregates from Tg brain when treated with Gla-OC for 3 days—amyloid of lesser density; Figure-Ga is HiLyte Aβ42 (10 μM) aggregated for 3 days where amyloid like morphology is evident. Figure Gb HiLyte Aβ42 (10 μM) exposed to Gla-OC (3 ng/ml) and aggregated for 3 days where amyloid is seen dispersed. FIG. 8H shows that osteocalcin binds to Abeta42 as evidenced by ELISA. FIG. 8I shows that Gla-OC reduces the toxicity of both native and mutant forms of Abeta42 as evidenced by lower activity of LDH in cell culture supernatant.

These results in tandem demonstrates that one of the mechanisms by which Gla-OC modulates amyloid beta42 is by binding to amyloid beta42 and inducing structural changes in the amyloid peptide which reduces toxic oligomeric confirmation and thereby cellular toxicity.

### Example 4

Examples Related to Effect Carboxylated Osteocalcin on Tau Pathology in db/db Mice

Mouse strain, db/db, was used to perform experiments related to Tau pathology. This is a congenic strain B6.BKS(D)-Lepr^{db/J} from Jackson Laboratory, Bar Harbor, Me. generated by backcrossing with black6 mice and selected on weight gain basis. Apart from obesity, hyperinsulinemia and metabolic dysfunction, db/db mouse exhibits Tau phosphorylation in hippocampal region [Kim B, Backus C, Oh S, Hayes J M, Feldman E L. 2009. Increased Tau phosphorylation and cleavage in mouse models of type 1 and type 2 diabetes. Endocrinology. 150: 5294-5301]. All experimental procedures in db/db mice were approved by the Institutional Animal Ethics Committee (IAEC/AQ/2016/150). For experimentation, mice were divided in below groups for treatment:
1. Wild type mice given vehicle (1×PBS; pH 7.4)
2. db/db mice given vehicle
3. db/db mice given carboxylated osteocalcin (Gla-OC) subcutaneously at dose, 300 per mouse of weight ranging from 30-32 g.
4. Wild type mice given Gla-OC (300 ng subcutaneously per mouse) Experimental duration was 15 days.

For the purposes of this study, wild type mice are labelled as Wt, untreated db/db mice is labelled as db/db, db/db mice treated with Gla-OC is labelled as db/db+Gla-OC and wild type mice treated with Gla-OC is labelled as Wt+Gla-OC. The treatments on db/db mice had no effect on its blood glucose level.

Isolation of hippocampus from mice: After harvesting the brain and washing it in saline, the brain was laid with dorsal side facing upwards. A scalpel blade was then employed to cut along the entire midline of the brain following the groove of the inter-hemispheric fissure. After gently pulling apart the two brain halves, each half was individually dissected by placing the half with the lateral side facing up. The posterior part of the brain, mid brain and the hindbrain were then cut off along the border of the cerebral cortex. Thereafter the olfactory bulb was removed. The tissue was then placed with the medial side facing up in order to remove the tissue covering the medial surface of the hippocampus. The spatula was inserted right below the corpus callosum and the thalamus, septum and underlying striatum were gently pulled out and cut away. The hippocampus, which is visible as a banana shaped structure was then carefully rolled out by holding the cerebral cortex down with one spatula and the other spatula under the ventral parts of the hippocampus. Tissue remnants were dissected away and the isolated hippocampus was homogenized in using RIPA lysis buffer containing protease-phosphatase inhibitor cocktail (Sigma-Aldrich, St Louis, Mo., USA). Protein estimation of brain homogenates was performed using Pierce BCA (bicinchoninic acid) protein assay kit and all the steps were performed as according to the manufacturer's instructions. For Western blot analysis, equal amounts of protein samples and pre-stained protein ladder were electrophoresed on 10-12% polyacrylamide (PAGE) gel using a BIO-RAD electrophoretic apparatus at 100 V. After run, the proteins were transferred to polyvinylidene difluoride membranes or PVDF (0.2 or 0.4 μm depending upon the size of the protein) using a Western transfer apparatus (90 V for 1.5 hours). The membranes were blocked for 1 hour using 5% BSA and incubated in primary antibody for 16-18 hours at 4° C. Tau phosphorylation and cleavage in brain were evaluated using specific antibodies. Phospho-Tau antibody (Ser199/202), a polyclonal antibody was procured from Thermo-Fisher, Wlatham, Mass., USA (Cat. No. 44-768G) and was used at dilution of 1:1000 and Tau5, a monoclonal antibody from Thermo-Fisher, Wlatham, Mass., USA (Cat. No AHB0042) was used at a dilution of 1:1000. After primary antibody incubation, membranes were washed with TBST for 15 mins (thrice) and then incubated with horse radish peroxidase (HRP) conjugated anti-rabbit or anti-mouse second antibodies (Cell Signaling Technology, Danvers, Mass., USA). The blots were again washed for 15 min with TBST (thrice). Immunoreactive proteins were visualized using chemiluminescence detection reagents (Bio-Rad) on LAS4000 imager (GE Healthcare Lifesciences, Marlborough, Mass., USA) using ImageQuant LAS4000 software.

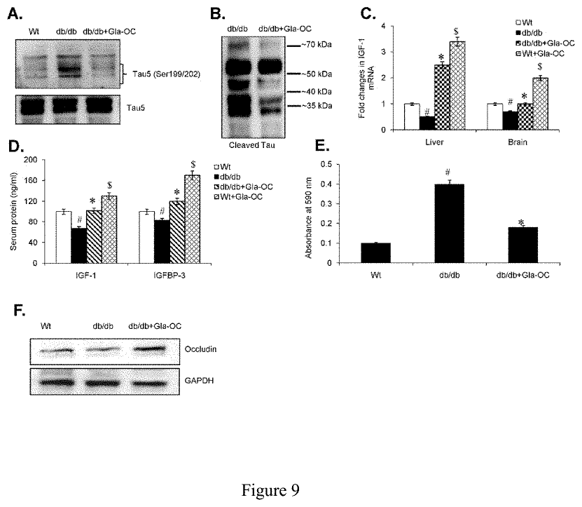

Result: FIG. 9A depicts Western blot that demonstrates the effect of Gla-OC on Tau phosphorylation. FIG. 9A depicts the inhibitory effect of Gla-OC on the phosphorylation of Tau protein in hippocampus of experimental mice, whilst FIG. 9B depicts the modulatory effect of Gla-OC on tau-cleavage. As can be observed, administration of Gla-OC reduces Tau cleavage and phosphorylation in hippocampus of db/db mice.

Effect of Gla-OC in blood brain barrier (BBB) protection: BBB is a semi-permeable membrane that separates the blood from cerebrospinal fluid. A growth factor that can enter the central nervous system or CNS by a saturable transport system at the BBB is insulin-like growth factor-1 (IGF-1). IGF-1 functions in synchrony with IGF binding proteins in the periphery to regulate the availability of IGF-1 to the CNS as well as slow down neuronal degeneration in some nervous system diseases [Pan W and Kastin A J, 2000, Interactions of IGF-1 with the blood brain barrier in vivo and in situ. Neuroendocrinology, 72: 171-178]. To determine the effect of Gla-OC on BBB, the levels of IGF-1 and IGFBP-3 (IGF binding protein-3) in serum were analyzed by ELISA. For this samples were applied on to high adsorbent 96-well flat bottom microtiter plates alongside recombinant standards (IGF-1 or IGFBP-3) at a concentration ranging from 0.001-0.1 ng and incubated for 2 h at 37° C. The unbound material was washed with phosphate buffered saline (pH 7.4) containing 0.2% Tween 20 (PBST), blocked with 10% BSA in PBST for 1 h at 37° C. After washing with PBST, 100 μl of primary antibodies at recommended dilutions was added per well and incubated for 3 h at 37° C. After washing with PBST, the wells were added with streptavidin horseradish peroxidise conjugated antibody (0.5 μg/ml) for 1 h and then treated with 3,30,5,50 tetramethylbenzidine (TMB) ready to use liquid substrate. Reaction was stopped using 100 μl of 1M HCl and the OD of each well was measured at 450 nm and 550 nm in an ELISA reader. 450 nm reading were subtracted from 550 nm values to correct imperfections in the microplate (Tecan microplate reader Infinite 200 PRO series, Switzerland with Magellan™ software). A curve fitting statistical software was used to plot a four-parameter logistic curve to calculate the results. The mRNA level of IGF-1 in liver and brain were evaluated using qPCR using primers mentioned in Table 1. In addition, uptake of the Evans Blue dye was also measured in the brain of experimental mice. Evans Blue dye test is a vascular permeability test based on the fact that albumin (to which Evans blue binds) does not cross the endothelial barrier. When a vascular permeability stimulus is present, either topically or systemically, blood vessels start to leak protein and thus, also the Evans blue that is bound to albumin. This results in a rapid bluish coloration of tissues that have permeable vessels. Herein the experiment is performed in accordance with Radu and Chernoff, 2013 [Radu M, Chernoff J. 2013. An in vivo assay to test blood vessel permeability. J Vis Exp. 16: e50062]. For the experiment 200 microlitre of 0.5% of Evans blue dye in PBS is injected into the tail vein of wild-type, db/db and db/db+Gla mice using 27-30 small gauge needle. After observation for 30 min, the mice are sacrificed and brains harvested. The brain samples are then weighed and placed in an eppendorf tubes to which 500 microlitre of formamide is added and incubated for 36 hours in a heating block set at 55° C. The mixture is centrifuged to remove tissue remnants and the Absorbance of the solvent is read at 610 nm using formamide as blank in TECAN microplate reader, Switzerland with Magellan™ software. The results are represented as nanogram of Evans blue per mg brain sample. Changes in protein expression of occludin, a tight junction marker was also tested in vessel fraction of the experimental mice by Western blot assay employing anti-occludin antibody, ab167161 from Abcam, Cambridge, UK. Cortical brain samples were cleaned of meninges and superficial blood vessels before brain homogenization and occludin estimation.

Results: FIG. 9C is a bar diagram showing changes in mRNA level of insulin like growth factor-1 (IGF-1) in the liver and brain of db/db mice treated with or without Gla-OC, as evidenced by qPCR. db/db mice showed reduced mRNA levels of IGF-1 in liver and brain of db/db mice. Gla-OC treatment induced IGF-1 mRNA level in liver of db/db mice and normalized the level of IGF-1 mRNA in brain. Significantly higher mRNA level of IGF-1 was also observed in the liver and brain of wild-type mice administered Gla-OC. FIG. 9D is a bar diagram showing changes in protein levels of IGF-1 and insulin like binding proteins-3 (IGFBP-3) as evidenced by ELISA in serum of db/db mice after Gla-OC treatment. db/db mice showed significantly lower level of IGF-1 and IGFBP-3 in serum as compared to wild-type. Gla-OC increased the level of IGF-1 and IGFBP-3 in wild-type mice. In db/db mice, normalization of IGF-1 and IGFBP-3 were seen upon Gla-OC treatment. IGFBP-3 was higher in Gla-OC+db/db mice than db/db control.

FIG. 9E indicates the difference in Evans Blue content in brain samples of Gla-OC treated db/db mice and control mice. The formamide solvent incubated with brain samples from Gla-OC treated db/db mice showed significantly lower amount of Evans blue dye accumulation as compared to that of db/db control. This shows that Evans blue dye permeability of endothelial barrier of Gla-OC treated db/db mice have reinstated the integrity. This was further examined at molecular level by evaluating the protein expression of a tight junction protein, occludin. FIG. 9F demonstrates higher expression of occludin protein in vessel fractions from Gla-OC treated animal than db/db mice. Collectively, the data obtained establishes the role of Gla-OC in reinstating the integrity of blood brain barrier.

### Example 5

### Examples Related to Effect of Amyloid 642 and Gla-OC on Osteoblast

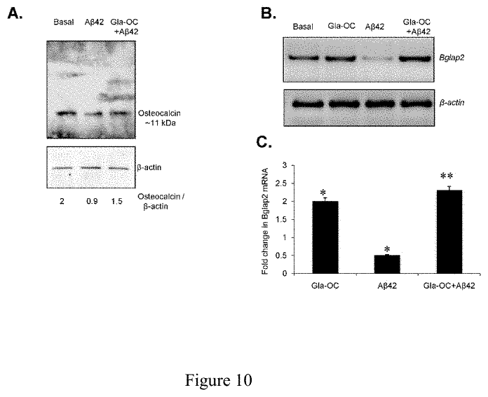

FIG. 10 demonstrates how Abeta42 inhibits osteocalcin expression in differentiating osteoblast cell cultures and how it is reversed with pre-treatment with Gla-OC (3 ng/ml). FIG. 10A is an immunoblot showing reduction in osteocalcin expression with Abeta42 addition and its reversal with Gla-OC treatment. Figure B demonstrates the inhibition in Bglap2 gene (mouse osteocalcin) with Abeta42 (1 micomolar) addition for 24 hours and reversal with pre-treatment with Gla-OC. FIG. 10C is a qPCR data that shows increased mRNA level of Bglap2 mRNA with pre-treatment with Gla-OC. Collectively the results demonstrate that Abeta42 is a cause for reduction in osteocalcin expression in osteoblasts and this can be countered with supplementation of Gla-OC.

Summary of Results:

From the data as obtained from the experimentation above, it is evident that Gla-OC is a potent modulator of diseases involving amyloid deposits. Significant clearance in the amyloid deposit is observed in 5×FAD mice treated with Gla-OC that is the prime reason for reduction in levels of Abeta42 in brain samples. The effect is deemed positive taking into account the improvement in other impaired cognitive function brought about by amyloidosis. The afore-mentioned effect of Gla-OC is validated by confirming the increase in the number and activity of astrocytes in the animals treated with Gla-OC. Elevation in the expression of amyloid uptake proteins like LRP-1 and CD36 and degradation of endocytosed Abeta42 by neprilysin and cathepsin is also observed upon Gla-OC treatment. These effects are brought about by the effect of Gla-OC on transcription factor EB. Another unexpected finding by the use of Gla-OC is increase in the level of Glu-OC in the circulation, which in turn is known to have neuroprotective function. Gla-OC also protects against Tau pathology as evidenced by reduction of Tau cleavage and phosphorylation in db/db mice. Gla-OC also improves the integrity of blood brain barrier via IGF-1.

Advantages of the present disclosure: The present disclosure provides with compositions that have been experimentally proven to be effective against clearance of pathogenic amyloids in various kinds of amyloid disorders. An aspect of the invention relates to, a composition comprising osteocalcin that has been found to be a modulator of diseases involving abnormal amyloid deposits. Abnormal amyloid deposits in brain is a result of aggregation of misfolded proteins that in turn leads to development of life-threatening disease condition. The composition binds to pathogenic amyloid, aids amyloid clearance, is non-toxic in nature and can be provided to patients suffering from amyloid disorders.

1. A composition comprising carboxylated osteocalcin as represented by SEQ ID NO: 1, for use in treatment of amyloid deposits.

2. The composition as claimed in claim 1 as represented by SEQ ID NO: 1, wherein treatment of abnormal amyloid deposits leads to treatment of diseases selected from the group consisting of type 2 diabetes mellitus, AL amyloidosis, secondary amyloidosis, familial amyloidosis, Alzheimer's disease, Down's syndrome, Idiopathic dialated cardiomyopathy, arthritis, tuberculosis, Lewy body variant of Alzheimer's, Parkinson dementia of Guam, spondylitis, Cerebral Amyloid Angiopathy (CAA) or congophilic angiopathy, Amyloidosis Dutch type, senile amyloid angiopathy, certain types of Creutzfeldt Jacob Disease, Kuru, fronto-temporal dementia with Parkinsonism linked to chromosome 17 (FTDP-17) caused by tau mutations, chronic traumatic encephalopathy, traumatic brain injury, Pick disease, corticobasal degeneration, dementia pugilistica and progressive supranuclear palsy.

3. The composition as claimed in claim 1, wherein the carboxylated osteocalcin as represented by SEQ ID NO: 1 is having fully carboxylated glutamic acid residues at positions 17, 21, and 24, and the osteocalcin is in calcium bound form.

4. The composition as claimed in claim 1, wherein the amyloid deposit is in a brain tissue or any tissue sample overexpressing pathogenic amyloid protein.

5. The composition as claimed in claim 1, wherein the carboxylated osteocalcin as represented by SEQ ID NO: 1 binds to both native and mutant forms of pathogenic amyloid protein and reduces its toxicity.

6. The composition as claimed in claim 1, wherein carboxylated osteocalcin as represented by SEQ ID NO: 1 induces clearance of amyloid deposits

7. The composition as claimed in claim 1, wherein the carboxylated osteocalcin as represented by SEQ ID NO: 1 increases the activity of phagocytic cells like glial cells, thereby leading to lowering/removal/clearance of abnormal or pathogenic amyloid deposits.

8. The composition as claimed in claim 1, wherein the carboxylated osteocalcin as represented by SEQ ID NO: 1 increases the expression of genes encoding neprilysin, low density like lipoprotein-1, cluster differentiation 36 or CD36, cathepsins, transcription factor EB and those associated with clearance of pathogenic peptides thereby leading to treatment of abnormal amyloid deposits.

9. A composition comprising carboxylated osteocalcin as represented by SEQ ID NO: 1, for use in protection of a tissue component in an amyloid diseased mammal, wherein the tissue component is blood brain barrier comprising endothelial cells.

10. The composition as claimed in claim 1, wherein carboxylated osteocalcin as represented by SEQ ID NO: 1 increases expression of IGF1 and IGF-1 binding proteins in circulation and reinstates the expression of tight junction protein, thereby maintaining the integrity of blood-brain barrier.

11. A composition comprising carboxylated osteocalcin as represented by SEQ ID NO: 1, for use in reducing cleavage and phosphorylation of abnormal Tau protein in brain.

12. A composition comprising carboxylated osteocalcin as represented by SEQ ID NO: 1, for use in increasing expression of undercarboxylated osteocalcin in circulation.

13. The composition as claimed in claim 1, further comprising at least one pharmaceutically acceptable excipient or carrier.

14. A method of treating a subject having disease involving abnormal amyloid deposits, said method comprising, administering to the subject a therapeutically effective amount of the composition as claimed in claim 1.
